(19) United States
(12) Patent Application Publication
(10) Pub. No.: US 2023/0118201 A1
(43) Pub. Date: Apr. 20, 2023

(54) CONDITIONING SHAMPOO COMPOSITION

(71) Applicant: The Procter & Gamble Company, Cincinnati, OH (US)

(72) Inventor: Brooke Michele Cochran, Cincinnati, OH (US)

(21) Appl. No.: 17/960,862

(22) Filed: Oct. 6, 2022

Related U.S. Application Data
(60) Provisional application No. 63/253,389, filed on Oct. 7, 2021.

(51) Int. Cl. A61K 8/44; A61Q 5/02; A61K 8/73; A61Q 5/00; A61Q 5/12; A61K 8/46

(52) U.S. Cl. CPC A61K 8/445; A61Q 5/02; A61K 8/737; A61Q 5/006; A61Q 5/12; A61K 8/466; A61K 2800/5426; A61K 2800/596

(57) ABSTRACT

A stable shampoo composition that contains a surfactant system with anionic and amphoteric surfactants, a cationic polymer having a charge density of 1.7 meq/g to 2.1 meq/g, and 1% to 1.5% inorganic salt.

### FIELD OF THE INVENTION

The present invention relates to a conditioning shampoo composition, in particular a conditioning shampoo composition with a cationic polymer having a charge density of 1.7 to 2.1 meq/g.

### BACKGROUND OF THE INVENTION

Historically, most commercial cleansing compositions, such as shampoo compositions, contain sulfate-based surfactant systems because they provide effective cleaning and a good user experience. Sulfate-based surfactant systems generally have acceptable viscosity making it easy to apply and distribute the shampoo composition throughout a user's hair. In addition, sulfate-based surfactant systems can generally be paired with cationic polymers that can form coacervate with the sulfate-based surfactant system during use thereby providing a shampoo with effective conditioning benefits.

Some consumers may prefer a shampoo composition that is substantially free of sulfate-based surfactant systems. These consumers may also prefer a higher conditioning shampoo because high conditioning shampoos generally feel less stripping to the hair. However, it can be difficult to formulate a shampoo with non-sulfate-based surfactants and cationic polymers that provides effective conditioning because the shampoo can be unstable. In particular, many shampoo compositions that contain anionic non-sulfate-based surfactants have a relatively high salt content that can cause an in situ coacervate phase to form in the composition prior to use (rather than the desired formation during use). The in situ coacervate can separate resulting in inconsistent in use performance and the product can appear cloudy and/or with a precipitated layer.

One way to prevent the in situ coacervate from forming prior to use is to decrease the salt concentration of the shampoo formulation. However, this can cause the viscosity of the shampoo composition to become too low, making it difficult to hold in a user's hand and apply to the hair and scalp. In these low salt formulations, the viscosity can be increased by decreasing the pH. However, many sulfate-free surfactant systems can hydrolyze at low pH resulting in viscosity and performance changes over time and will eventually lead to phase separation.

Therefore, there is a need for a stable shampoo product with a sufficient viscosity and superior product performance that contains one or more non-sulfated anionic surfactants, cationic polymers, and inorganic salts without forming the in situ coacervate phase in the product prior to dilution with water.

### SUMMARY OF THE INVENTION

A stable shampoo composition comprising: (a) a surfactant system comprising: (i) 3% to 35% of an anionic surfactant; (ii) 5% to 15% of an amphoteric surfactant; (b) 0.01% to 2% of a cationic polymer having a charge density of 1.7 meq/g to 2.1 meq/g; (c) 1 to 1.5% inorganic salt; wherein the composition is substantially free of sulfated surfactants.

A stable shampoo composition comprising: (a) a surfactant system comprising: (i) 3% to 35% of an anionic surfactant selected from isethionates, sarcosinates, and combinations thereof; (ii) 5% to 15% of an amphoteric surfactant selected from cocamidopropyl betaine, lauramidopropyl betaine, and combinations thereof; wherein the ratio of the anionic to the amphoteric surfactant is 0.5:1 to 1.5:1; (a) 0.01% to 2% of a cationic polymer having a charge density of 1.7 meq/g to 2.1 meq/g; wherein the cationic surfactant is selected from hyroxypropyltrimonium guar, Polyquaternium 10, and combinations thereof; (b) 1% to 1.5% sodium chloride; wherein the composition is substantially free of sulfated surfactants; wherein the shampoo composition lacks in situ coacervate, as determined by the Microscopy Method to Determine Lack of In Situ Coacervate.

### BRIEF DESCRIPTION OF THE DRAWINGS

FIG. 1 is a 20× micrograph of a shampoo composition that contains in situ coacervate.

FIG. 2 is a 10× micrograph of the shampoo composition of FIG. 1.

FIG. 3 is a photograph of Comparative Example 4, nine months after making

### DETAILED DESCRIPTION OF THE INVENTION

Shampoo compositions can contain cationic polymers that can provide a conditioning benefit. Consumers, especially those who use shampoo compositions that are substantially free of sulfate-based surfactants, generally prefer these conditioning shampoos because they often feel less stripping to the hair. However, it can be difficult to formulate a shampoo with these cationic polymers because the shampoo can be unstable and form undesired coacervate in the bottle (referred to herein as “in situ coacervate” or an “in situ coacervate phase”, which is a coacervate that forms in the composition, prior to dilution), as opposed to when it is diluted with water when a user washes their hair.

The formation of coacervate upon dilution of the cleansing composition with water during use, rather than while in the bottle on the shelf, is important to improving wet conditioning and deposition of various conditioning actives, especially those that have small droplet sizes (i.e., ≤2 microns). One way to form good quality coacervate at the right time (upon dilution during use), is to formulate with very low (e.g., <1%) or no salt by limiting the amount of inorganic salt that is added to the composition and that comes in with the surfactant materials. However, inorganic salt helps to elongate micelles to build viscosity. Therefore, these compositions generally have a viscosity that is too low, which is not consumer preferred because it is difficult to use the product.

It was found that a stable shampoo composition with an acceptable viscosity and product performance could be made with 1% to 1.5% total inorganic salt content if a conditioning polymer with a density of 1.7 to 2.1 meq/gm was used.

It was found that formulas containing this higher level of inorganic salt (e.g., 1% to 1.5% total inorganic salt) had a higher viscosity than similar formulas that contained lower levels of inorganic salt or formulas that were substantially free or free of inorganic salt. This results from more elongation of surfactant micelles (see Robbins, Clarence. Chemical and Physical Behavior of Human Hair, Springer, Berlin, Germany, 2012, pp. 335. “To control the viscosity of many shampoos, salt is added to the surfactant system. The interaction between salt and long chain surfactants transforms the small spherical micelles of the surfactants into larger rod-like . . . structures that increase the viscosity of the liquid shampoo.”) These higher viscosity formulas may be consumer preferred because it is easier to apply across a user's hair and scalp without it running through their fingers.

Another benefit of the higher viscosity shampoo composition is that since there is more elongation of surfactant micelles, a broader range of formulas with acceptable viscosity can be designed because other formula ingredients are not required to build viscosity. For example, viscosity modifiers, other than an inorganic salt, may not be needed. The composition may be free of or substantially free of viscosity modifiers, other than inorganic salt (e.g., sodium chloride, potassium chloride, sodium sulfate, ammonium chloride, sodium bromide, and combinations thereof), which can include carbomers, cross-linked acrylates, hydrophobically modified associative polymers and cellulose, as described in US Pub. Nos. 2019/0105246 and 2019/010524, incorporated by reference. This can make the shampoo easier to distribute across a user's hair and scalp.

Because acceptable viscosity can be achieved using inorganic salt within the range of 1% to 1.5% total inorganic salt content, formulas can be made at a higher pH, which can make the composition more stable and effective due to less surfactant hydrolysis resulting in more consistent viscosity and performance over time.

It can be difficult to formulate stable compositions that include 1 to 1.5% total inorganic salt because inorganic salt in the shampoo composition can come from raw materials and can be added to the formulations. For example, amphoteric surfactants such as betaines typically come with high levels of inorganic salt such as sodium chloride. Use high-salt-containing raw materials at levels that bring in greater than 1.5% inorganic salt when summed with added inorganic salt can result in in situ coacervate.

It was found that if the composition contained a polymer with charge density of 1.7 to 2.1 meq/g and 1% to 1.5% total inorganic salt, then the shampoo composition could be stable and can also have a consumer acceptable viscosity and conditioning performance. The cationic polymer and the organic salt can be balanced to maintain a stable solution. If too much salt is added, the polymer can form an in situ coacervate before being diluted with water during use.

The pH can be 4 to 8, alternatively 4.5 to 7.5, alternatively 5 to 7, alternatively 5.5 to 6.5, alternatively 5.5 to 6, and alternatively 6 to 6.5, as determined by the pH Test Method, described herein.

The shampoo composition can include 0.75% to 1.5% inorganic salt, alternatively 0.8% to 1.4%, alternatively 0.9% to 1.4%. The inorganic salt can be an inorganic chloride salt. The wt. % inorganic chloride salt can be determined by the Argentometry Method to Measure wt % Inorganic Chloride Salt Test Method, described herein. The inorganic salt can be a viscosity modifier. The shampoo composition can contain no viscosity modifier other than one or more inorganic salts.

The shampoo composition can have a viscosity of 3000 cP to 20,000 cP, alternatively 4000 cP to 15,000 cP, alternatively from 4500 cP to 12,000 cP, alternatively 5,000 cP to 11,000 cP, and alternatively 7,000 cP to 10,000 cP, as measured at 26.6° C., as measured by the Cone/Plate Viscosity Measurement Test Method, described herein.

The shampoo composition can be used to clean and condition hair. First, the user dispenses the liquid shampoo composition from the bottle into their hand or onto a cleaning implement. Then, they massage the shampoo into their wet hair. While they are massaging the shampoo composition into the hair the shampoo is diluted and a coacervate can form and the shampoo can lather. After massaging into hair, the shampoo composition is rinsed from the user's hair and at least a portion of the cationic polymers can be deposited on the user's hair, which can provide a conditioning benefit. Shampooing can be repeated, if desired, and/or a conditioner can be applied. The conditioner can be a rinse-off conditioner or a leave-in conditioner.

It may be consumer desirable to have a shampoo composition with a minimal level of ingredients. The shampoo composition can be formulated without polymeric thickeners or suspending agents such as carbomer, EGDS or thixcin. The shampoo composition may be comprised of 11 or fewer ingredients, 10 or fewer ingredients, 9 or fewer ingredients, 8 or fewer ingredients, 7 or fewer ingredients, 6 or fewer ingredients. The minimal ingredient formula can include water, anionic surfactant, amphoteric surfactant, cationic polymer, inorganic salt, and perfume. It is understood that perfumes can be formed from one or more materials. In some examples, the composition can be free of or substantially free of fragrance. In another example, the composition can be free of or substantially free of PEG.

While the specification concludes with claims particularly pointing out and distinctly claiming the invention, it is believed that the present disclosure will be better understood from the following description.

As used herein, “cleansing composition” includes personal cleansing products such as shampoos, conditioners, conditioning shampoos, shower gels, liquid hand cleansers, facial cleansers, and other surfactant-based liquid compositions.

The shampoo composition can be clear prior to dilution with water. The term “clear” or “transparent” as used herein, means that the compositions have a percent transparency (%T) of at least 80% transmittance at 600 nm. The %T can be at 600 nm 75% to 100%, 80% to 100%, 85% to 100%, 90% to 100%, 95% to 100%.

As used herein, the term “fluid” includes liquids and gels.

As used herein, “molecular weight” or “M·Wt.” refers to the weight average molecular weight unless otherwise stated. Molecular weight is measured using industry standard method, gel permeation chromatography (“GPC”). The molecular weight has units of grams/mol.

As used herein, “substantially free” refers to less than 0.5%, alternatively less than 0.25%, alternatively less than 0.1%, alternatively less than 0.05%, alternatively less than 0.02%, and alternatively less than 0.01%.

As used herein, “sulfate free” and “substantially free of sulfates” means essentially free of sulfate-containing compounds except as otherwise incidentally incorporated as minor components. Sulfate free contains no detectable sulfated surfactants.

As used herein, “sulfated surfactants” or “sulfate-based surfactants” means surfactants which contain a sulfate group. The term “substantially free of sulfated surfactants” or “substantially free of sulfate-based surfactants” means essentially free of surfactants containing a sulfate group except as otherwise incidentally incorporated as minor components.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

Unless otherwise noted, all component or composition levels are in reference to the active portion of that component or composition, and are exclusive of impurities, for example, residual solvents or by-products, which may be present in commercially available sources of such components or compositions.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Surfactant

The cleansing compositions described herein can include one or more surfactants in the surfactant system. The one or more surfactants can be substantially free of sulfate-based surfactants. As can be appreciated, surfactants provide a cleaning benefit to soiled articles such as hair, skin, and hair follicles by facilitating the removal of oil and other soils. Surfactants generally facilitate such cleaning due to their amphiphilic nature which allows for the surfactants to break up, and form micelles around, oil and other soils which can then be rinsed out, thereby removing them from the soiled article. Suitable surfactants for a cleansing composition can include anionic moieties to allow for the formation of a coacervate with a cationic polymer. The surfactant can be selected from anionic surfactants, amphoteric surfactants, zwitterionic surfactants, non-ionic surfactants, and combinations thereof.

Cleansing compositions typically employ sulfate-based surfactant systems (such as, but not limited to, sodium lauryl sulfate) because of their effectiveness in lather production, stability, clarity and cleansing. The cleansing compositions described herein are substantially free of sulfate-based surfactants. “Substantially free” of sulfate based surfactants as used herein means 0 wt % to 3 wt %, alternatively 0 wt % to 2 wt %, alternatively 0 wt % to 1 wt %, alternatively 0 wt % to 0.5 wt %, alternatively 0 wt % to 0.25 wt %, alternatively 0 wt % to 0.1 wt %, alternatively 0 wt % to 0.05 wt %, alternatively 0 wt % to 0.01 wt %, alternatively 0 wt % to 0.001 wt %, and/or alternatively free of sulfates. As used herein, “free of” means 0 wt %.

Additionally, the surfactants can be added to the composition as a solution, instead of the neat material and the solution can include inorganic salts that can be added to the formula. The surfactant formula can have inorganic salt that can be 0% to 2% of inorganic salts of the final composition, alternatively 0.1% to 1.5%, and alternatively 0.2% to 1%.

Suitable surfactants that are substantially free of sulfates can include sodium, ammonium or potassium salts of isethionates; sodium, ammonium or potassium salts of sulfonates; sodium, ammonium or potassium salts of ether sulfonates; sodium, ammonium or potassium salts of sulfosuccinates; sodium, ammonium or potassium salts of sulfoacetates; sodium, ammonium or potassium salts of glycinates; sodium, ammonium or potassium salts of sarcosinates; sodium, ammonium or potassium salts of glutamates; sodium, ammonium or potassium salts of alaninates; sodium, ammonium or potassium salts of carboxylates; sodium, ammonium or potassium salts of taurates; sodium, ammonium or potassium salts of phosphate esters; and combinations thereof.

The concentration of the surfactant in the composition should be sufficient to provide the desired cleaning and lather performance The cleansing composition can include a total surfactant level of 5% to 50%, alternatively 8% to 40%, alternatively 10% to 30%, alternatively 12% to 25%, alternatively 13% to 23%, alternatively 14% to 21%, alternatively 15% to 20%.

The cleansing composition can include 3% to 30% anionic surfactant, alternatively 4% to 20%, alternatively 5% to 15%, alternatively 6% to 12%, and alternatively 7% to 10%. The cleansing composition can include 3% to 40% amphoteric surfactant, alternatively 4% to 30%, alternatively 5% to 25%, alternatively 6% to 18%, alternatively 7% to 15%, alternatively 8% to 13%, and alternatively 9% to 11%.

The ratio of anionic surfactant to amphoteric surfactant can be 0.4:1 to 1.25:1, alternatively 0.5:1 to 1.1:1, and alternatively 0.6:1 to 1:1. In some examples, the ratio of anionic surfactant to amphoteric surfactant is less than 1.1:1, and alternatively less than 1:1.

In some examples, inorganic salt is added to the shampoo composition with the surfactant raw materials. In one example, the surfactant raw materials include less than 1.5% inorganic salt, alternatively less than 1.25%, alternatively less than 1%, alternatively less than 0.7%, alternatively less than 0.5%, alternatively less than 0.25%, alternatively less than 0.2%, alternatively less than 0.15%, alternatively less than or equal to 0.1%. In some examples, at least 0.05% inorganic salt is added to the formula via the surfactant raw materials, alternatively at least 0.07%, and alternatively at least 0.1%.

The surfactant system can include one or more amino acid based anionic surfactants. Non-limiting examples of amino acid based anionic surfactants can include sodium, ammonium or potassium salts of acyl glycinates; sodium, ammonium or potassium salts of acyl sarcosinates; sodium, ammonium or potassium salts of acyl glutamates; sodium, ammonium or potassium salts of acyl alaninates and combinations thereof.

The amino acid based anionic surfactant can be a glutamate, for instance an acyl glutamate. Non-limiting examples of acyl glutamates can be selected from the group consisting of sodium cocoyl glutamate, disodium cocoyl glutamate, ammonium cocoyl glutamate, diammonium cocoyl glutamate, sodium lauroyl glutamate, disodium lauroyl glutamate, sodium cocoyl hydrolyzed wheat protein glutamate, disodium cocoyl hydrolyzed wheat protein glutamate, potassium cocoyl glutamate, dipotassium cocoyl glutamate, potassium lauroyl glutamate, dipotassium lauroyl glutamate, potassium cocoyl hydrolyzed wheat protein glutamate, dipotassium cocoyl hydrolyzed wheat protein glutamate, sodium capryloyl glutamate, disodium capryloyl glutamate, potassium capryloyl glutamate, dipotassium capryloyl glutamate, sodium undecylenoyl glutamate, disodium undecylenoyl glutamate, potassium undecylenoyl glutamate, dipotassium undecylenoyl glutamate, disodium hydrogenated tallow glutamate, sodium stearoyl glutamate, disodium stearoyl glutamate, potassium stearoyl glutamate, dipotassium stearoyl glutamate, sodium myristoyl glutamate, disodium myristoyl glutamate, potassium myristoyl glutamate, dipotassium myristoyl glutamate, sodium cocoyl/hydrogenated tallow glutamate, sodium cocoyl/palmoyl/sunfloweroyl glutamate, sodium hydrogenated tallowoyl Glutamate, sodium olivoyl glutamate, disodium olivoyl glutamate, sodium palmoyl glutamate, disodium palmoyl Glutamate, TEA-cocoyl glutamate, TEA-hydrogenated tallowoyl glutamate, TEA-lauroyl glutamate, and mixtures thereof.

The amino acid based anionic surfactant can be an alaninate, for instance an acyl alaninate. Non-limiting example of acyl alaninates can include sodium cocoyl alaninate, sodium lauroyl alaninate, sodium N-dodecanoyl-l-alaninate and combination thereof.

The amino acid based anionic surfactant can be a sarcosinate, for instance an acyl sarcosinate. Non-limiting examples of sarcosinates can be selected from the group consisting of sodium lauroyl sarcosinate, sodium cocoyl sarcosinate, sodium myristoyl sarcosinate, TEA-cocoyl sarcosinate, ammonium cocoyl sarcosinate, ammonium lauroyl sarcosinate, dimer dilinoleyl bis-lauroylglutamate/lauroylsarcosinate, disodium lauroamphodiacetate lauroyl sarcosinate, isopropyl lauroyl sarcosinate, potassium cocoyl sarcosinate, potassium lauroyl sarcosinate, sodium cocoyl sarcosinate, sodium lauroyl sarcosinate, sodium myristoyl sarcosinate, sodium oleoyl sarcosinate, sodium palmitoyl sarcosinate, TEA-cocoyl sarcosinate, TEA-lauroyl sarcosinate, TEA-oleoyl sarcosinate, TEA-palm kernel sarcosinate, and combinations thereof.

The amino acid based anionic surfactant can be a glycinate for instance an acyl glycinate. Non-limiting example of acyl glycinates can include sodium cocoyl glycinate, sodium lauroyl glycinate and combination thereof.

The composition can contain additional anionic surfactants selected from the group consisting of sulfosuccinates, isethionates, sulfonates, sulfoacetates, glucose carboxylates, alkyl ether carboxylates, acyl taurates, and mixture thereof.

Non-limiting examples of sulfosuccinate surfactants can include disodium N-octadecyl sulfosuccinate, disodium lauryl sulfosuccinate, diammonium lauryl sulfosuccinate, sodium lauryl sulfosuccinate, disodium laureth sulfosuccinate, tetrasodium N-(1,2-dicarboxyethyl)-N-octadecyl sulfosuccinnate, diamyl ester of sodium sulfosuccinic acid, dihexyl ester of sodium sulfosuccinic acid, dioctyl esters of sodium sulfosuccinic acid, and combinations thereof. The composition can comprise a sulfosuccinate level 2% to 22%, by weight, 3% to 19%, by weight, 4% to 17%, by weight, and/or 5% to 15%, by weight.

Suitable isethionate surfactants can include the reaction product of fatty acids esterified with isethionic acid and neutralized with sodium hydroxide. Suitable fatty acids for isethionate surfactants can be derived from coconut oil or palm kernel oil including amides of methyl tauride. Non-limiting examples of isethionates can be selected from the group consisting of sodium lauroyl methyl isethionate, sodium cocoyl isethionate, ammonium cocoyl isethionate, sodium hydrogenated cocoyl methyl isethionate, sodium lauroyl isethionate, sodium cocoyl methyl isethionate, sodium myristoyl isethionate, sodium oleoyl isethionate, sodium oleyl methyl isethionate, sodium palm kerneloyl isethionate, sodium stearoyl methyl isethionate, and mixtures thereof.

Non-limiting examples of sulfonates can include alpha olefin sulfonates, linear alkylbenzene sulfonates, sodium laurylglucosides hydroxypropylsulfonate and combination thereof.

Non-limiting examples of sulfoacetates can include sodium lauryl sulfoacetate, ammonium lauryl sulfoacetate and combination thereof.

Non-limiting example of glucose carboxylates can include sodium lauryl glucoside carboxylate, sodium cocoyl glucoside carboxylate and combinations thereof.

Non-limiting example of alkyl ether carboxylate can include sodium laureth-4 carboxylate, laureth-5 carboxylate, laureth-13 carboxylate, sodium C12-13 pareth-8 carboxylate, sodium C12-15 pareth-8 carboxylate and combination thereof.

Non-limiting example of acyl taurates can include sodium methyl cocoyl taurate, sodium methyl lauroyl taurate, sodium methyl oleoyl taurate, sodium caproyl methyl taurate, and combination thereof.

The surfactant system may further comprise one or more amphoteric surfactants and the amphoteric surfactant can be selected from the group consisting of betaines, sultaines, hydroxysultanes, amphohydroxypropyl sulfonates, alkyl amphoactates, alkyl amphodiacetates, alkyl amphopropionates and combination thereof.

Examples of betaine amphoteric surfactants can include coco dimethyl carboxymethyl betaine, cocoamidopropyl betaine (CAPB), cocobetaine, lauryl amidopropyl betaine (LAPB), oleyl betaine, lauryl dimethyl carboxymethyl betaine, lauryl dimethyl alphacarboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, lauryl bis-(2-hydroxypropyl)alpha-carboxyethyl betaine, cetyl betaine, and mixtures thereof. Examples of sulfobetaines can include coco dimethyl sulfopropyl betaine, stearyl dimethyl sulfopropyl betaine, lauryl dimethyl sulfoethyl betaine, lauryl bis-(2-hydroxyethyl) sulfopropyl betaine and mixtures thereof.

Non-limiting example of alkylamphoacetates can include sodium cocoyl amphoacetate, sodium lauroyl amphoacetate and combination thereof.

The amphoteric surfactant can comprise cocamidopropyl betaine (CAPB), lauramidopropyl betaine (LAPB), and combinations thereof.

The surfactant system may further comprise one or more non-ionic surfactants and the non-ionic surfactant can be selected from the group consisting alkyl polyglucoside, alkyl glycoside, acyl glucamide and mixture thereof. Non-limiting examples of alkyl glucosides can include decyl glucoside, cocoyl glucoside, lauroyl glucoside and combination thereof.

Non-limiting examples of acyl glucamide can include lauroyl/myristoyl methyl glucamide, capryloyl/caproyl methyl glucamide, lauroyl/myristoyl methyl glucamide, cocoyl methyl glucamide and combinations thereof.

The composition can contain a non-ionic detersive surfactants that can include cocamide, cocamide methyl MEA, cocamide DEA, cocamide MEA, cocamide MIPA, lauramide DEA, lauramide MEA, lauramide MIPA, myristamide DEA, myristamide MEA, PEG-20 cocamide MEA, PEG-2 cocamide, PEG-3 cocamide, PEG-4 cocamide, PEG-5 cocamide, PEG-6 cocamide, PEG-7 cocamide, PEG-3 lauramide, PEG-5 lauramide, PEG-3 oleamide, PPG-2 cocamide, PPG-2 hydroxyethyl cocamide, and mixtures thereof.

### Cationic Polymer

A cleansing composition can include a cationic polymer to allow formation of a coacervate.

As can be appreciated, the cationic charge of a cationic polymer can interact with an anionic charge of a surfactant to form the coacervate. Suitable cationic polymers can include: (a) a cationic guar polymer, (b) a cationic non-guar galactomannan polymer, (c) a cationic starch polymer, (d) a cationic copolymer of acrylamide monomers and cationic monomers, (e) a synthetic, non-crosslinked, cationic polymer, which may or may not form lyotropic liquid crystals upon combination with the detersive surfactant, and (f) a cationic cellulose polymer. In certain examples, more than one cationic polymer can be included. In some examples, the cationic polymer can include polyquatemium-10, guar hydroxypropyltrimonium chloride, polyquaternium-6, and combinations thereof.

The charge density can be greater than 1.5 meq/g, alternatively greater than 1.6 meq/g, and alternatively greater than 1.7 meq/g. The charge density can be 1.5 meq/g to 3 meq/g, alternatively 1.55 meq/g to 2.8 meq/g, alternatively 1.6 meq/g to 2.6 meq/g, alternatively 1.65 meq/g to 2.4 meq/g, alternatively 1.7 meq/g to 2.2 meq/g, alternatively 1.75 meq/g to 2.15 meq/g, and alternatively 1.8 meq/g to 2.1 meq/g.

A cationic polymer can be included by weight of the cleansing composition at 0.05% to 3%, alternatively 0.075% to 2.0%, alternatively 0.1% to 1.0%, alternatively 0.1% to 0.75%, alternatively 0.12% to 0.5%, and alternatively 0.15% to 0.35%. The charge densities can be measured at the pH of intended use of the cleansing composition. (e.g., at pH 3 to pH 9; or pH 4 to pH 8). The average molecular weight of cationic polymers can generally be between 10,000 and 10 million, between 50,000 and 5 million, and between 100,000 and 3 million, and between 300,000 and 3 million and between 100,000 and 2.5 million. Low molecular weight cationic polymers can be used. Low molecular weight cationic polymers can have greater translucency in the liquid carrier of a cleansing composition. The cationic polymer can be a single type, such as the cationic guar polymer guar hydroxypropyltrimonium chloride having a weight average molecular weight of 2.5 million g/mol or less, and the cleansing composition can have an additional cationic polymer of the same or different types.

Charge density of cationic polymers other than cationic guar polymers can be determined by measuring % Nitrogen. % Nitrogen is measured using USP <461> Method II. % Nitrogen can then be converted to Cationic Polymer Charge Density by calculations known in the art.

The charge density of cationic guar polymers can be calculated as follows: first, calculate the degree of substitution, as disclosed in WO 2019/096601, page 3, lines 4-22, and then cationic charge density can be calculated from the degree of substitution, as described in WO 2013/011122, page 8, lines 8-17, the disclosure of these publications are incorporated by reference.

### Cationic Guar Polymer

The cationic polymer can be a cationic guar polymer, which is a cationically substituted galactomannan (guar) gum derivative. Suitable guar gums for guar gum derivatives can be obtained as a naturally occurring material from the seeds of the guar plant. As can be appreciated, the guar molecule is a straight chain mannan which is branched at regular intervals with single membered galactose units on alternative mannose units. The mannose units are linked to each other by means of β(1-4) glycosidic linkages. The galactose branching arises by way of an α(1-6) linkage. Cationic derivatives of the guar gums can be obtained through reactions between the hydroxyl groups of the polygalactomannan and reactive quaternary ammonium compounds. The degree of substitution of the cationic groups onto the guar structure can be sufficient to provide the requisite cationic charge density described above.

A cationic guar polymer can have a weight average molecular weight (“M·Wt.”) of less than 3 million g/mol, and can have a charge density 0.05 meq/g to 2.5 meq/g. Alternatively, the cationic guar polymer can have a weight average M·Wt. of less than 1.5 million g/mol, 150 thousand g/mol to 1.5 million g/mol, 200 thousand g/mol to 1.5 million g/mol, 300 thousand g/mol to 1.5 million g/mol, and 700,000 thousand g/mol to 1.5 million g/mol. The cationic guar polymer can have a charge density 1.7 meq/g to 2.1 meq/g.

A cleansing composition can include 0.01% to less than 0.7%, by weight of the cleansing composition of a cationic guar polymer, 0.04% to 0.55%, by weight, 0.08% to 0.5%, by weight, 0.16% to 0.5%, by weight, 0.2% to 0.5%, by weight, 0.3% to 0.5%, by weight, and 0.4% to 0.5%, by weight.

The cationic guar polymer can be formed from quaternary ammonium compounds which conform to general Formula II:

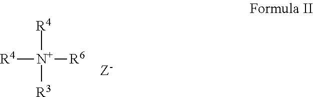

wherein where R³, R⁴ and R⁵ are methyl or ethyl groups; and R⁶ is either an epoxyalkyl group of the general Formula III:

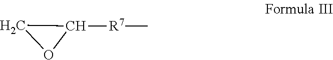

or R⁶ is a halohydrin group of the general Formula IV:

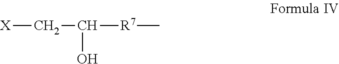

wherein R⁷ is a C₁ to C₃ alkylene; X is chlorine or bromine, and Z is an anion such as Cl—, Br—, I— or HSO₄—.

Suitable cationic guar polymers can conform to the general formula V:

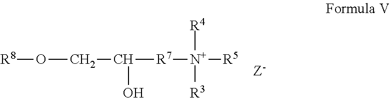

wherein R⁸ is guar gum; and wherein R⁴, R⁵, R⁶ and R⁷ are as defined above; and wherein Z is a halogen. Suitable cationic guar polymers can conform to Formula VI:

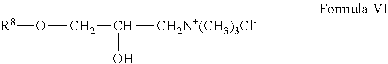

wherein R⁸ is guar gum.

Suitable cationic guar polymers can also include cationic guar gum derivatives, such as guar hydroxypropyltrimonium chloride. Suitable examples of guar hydroxypropyltrimonium chlorides can include the Jaguar® series commercially available from Solvay® S.A., Hi-Care Series from Rhodia®, and N-Hance™ and AquaCat™ from Ashland™ Inc. For example, N-Hance™ BF-17 is a borate (boron) free guar polymers. N-Hance™ BF-17 has a charge density of 1.7 meq/g and M·Wt. of 800,000. BF-17 has a charge density of 1.7 meq/g and M·Wt. of 800,000. BF-17 has a charge density of 1.7 meq/g and M·Wt. of 800,000. BF-17 has a charge density of 1.7 meq/g and M·Wt. of 800,000. BF-17 has a charge density of 1.7 meq/g and M·Wt. of 800,000.

### Cationic Non-Guar Galactomannan Polymer

The cationic polymer can be a galactomannan polymer derivative. Suitable galactomannan polymer can have a mannose to galactose ratio of greater than 2:1 on a monomer to monomer basis and can be a cationic galactomannan polymer derivative or an amphoteric galactomannan polymer derivative having a net positive charge. As used herein, the term “cationic galactomannan” refers to a galactomannan polymer to which a cationic group is added. The term “amphoteric galactomannan” refers to a galactomannan polymer to which a cationic group and an anionic group are added such that the polymer has a net positive charge.

Galactomannan polymers can be present in the endosperm of seeds of the Leguminosae family Galactomannan polymers are made up of a combination of mannose monomers and galactose monomers. The galactomannan molecule is a straight chain mannan branched at regular intervals with single membered galactose units on specific mannose units. The mannose units are linked to each other by means of β (1-4) glycosidic linkages. The galactose branching arises by way of an α (1-6) linkage. The ratio of mannose monomers to galactose monomers varies according to the species of the plant and can be affected by climate. Non Guar Galactomannan polymer derivatives can have a ratio of mannose to galactose of greater than 2:1 on a monomer to monomer basis. Suitable ratios of mannose to galactose can also be greater than 3:1 or greater than 4:1. Analysis of mannose to galactose ratios is well known in the art and is typically based on the measurement of the galactose content.

The gum for use in preparing the non-guar galactomannan polymer derivatives can be obtained from naturally occurring materials such as seeds or beans from plants. Examples of various non-guar galactomannan polymers include Tara gum (3 parts mannose/1 part galactose), Locust bean or Carob (4 parts mannose/1 part galactose), and Cassia gum (5 parts mannose/1 part galactose).

A non-guar galactomannan polymer derivative can have a M. Wt. 1,000 g/mol to 10,000,000 g/mol, and a M·Wt. 5,000 g/mol to 3,000,000 g/mol.

The cleansing compositions described herein can include galactomannan polymer derivatives which have a cationic charge density 1.7 meq/g to 2.1 meq/g. The galactomannan polymer derivatives can have a cationic charge density 1.7 meq/g to 2.1 meq/g. The degree of substitution of the cationic groups onto the galactomannan structure can be sufficient to provide the requisite cationic charge density.

A galactomannan polymer derivative can be a cationic derivative of the non-guar galactomannan polymer, which is obtained by reaction between the hydroxyl groups of the polygalactomannan polymer and reactive quaternary ammonium compounds. Suitable quaternary ammonium compounds for use in forming the cationic galactomannan polymer derivatives include those conforming to the general Formulas II to VI, as defined above.

Cationic non-guar galactomannan polymer derivatives formed from the reagents described above can be represented by the general Formula VII:

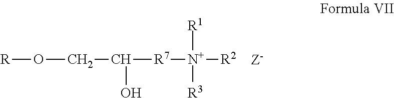

wherein R is the gum. The cationic galactomannan derivative can be a gum hydroxypropyltrimethylammonium chloride, which can be more specifically represented by the general Formula VIII:

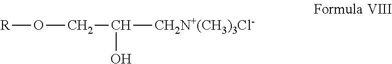

The galactomannan polymer derivative can be an amphoteric galactomannan polymer derivative having a net positive charge, obtained when the cationic galactomannan polymer derivative further comprises an anionic group.

A cationic non-guar galactomannan can have a ratio of mannose to galactose which is greater than 4:1, a M·Wt. of 100,000 g/mol to 500,000 g/mol, a M·Wt. of 50,000 g/mol to 400,000 g/mol, and a cationic charge density 1.7 meq/g to 2.1 meq/g.

Cleansing compositions can include at least 0.05% of a galactomannan polymer derivative by weight of the composition. The cleansing compositions can include 0.05% to 2%, by weight of the composition, of a galactomannan polymer derivative.

### Cationic Starch Polymers

Suitable cationic polymers can also be water-soluble cationically modified starch polymers. As used herein, the term “cationically modified starch” refers to a starch to which a cationic group is added prior to degradation of the starch to a smaller molecular weight, or wherein a cationic group is added after modification of the starch to achieve a desired molecular weight. The definition of the term “cationically modified starch” also includes amphoterically modified starch. The term “amphoterically modified starch” refers to a starch hydrolysate to which a cationic group and an anionic group are added.

The cleansing compositions described herein can include cationically modified starch polymers at a range of 0.01% to 10%, and/or 0.05% to 5%, by weight of the composition. The cationically modified starch polymers disclosed herein have a percent of bound nitrogen of 0.5% to 4%.

The cationically modified starch polymers can have a molecular weight 850,000 g/mol to 15,000,000 g/mol and 900,000 g/mol to 5,000,000 g/mol.

Suitable cationically modified starch polymers can have a charge density of 1.7 meq/g to 2.1 meq/g. The chemical modification to obtain such a charge density can include the addition of amino and/or ammonium groups into the starch molecules. Non-limiting examples of such ammonium groups can include substituents such as hydroxypropyl trimmonium chloride, trimethylhydroxypropyl ammonium chloride, dimethylstearylhydroxypropyl ammonium chloride, and dimethyldodecylhydroxypropyl ammonium chloride. Further details are described in Solarek, D. B., Cationic Starches in Modified Starches: Properties and Uses, Wurzburg, O. B., Ed., CRC Press, Inc., Boca Raton, Fla. 1986, pp 113-125 which is hereby incorporated by reference. The cationic groups can be added to the starch prior to degradation to a smaller molecular weight or the cationic groups may be added after such modification.

A cationically modified starch polymer can have a degree of substitution of a cationic group 0.2 to 2.5. As used herein, the “degree of substitution” of the cationically modified starch polymers is an average measure of the number of hydroxyl groups on each anhydroglucose unit which is derivatized by substituent groups. Since each anhydroglucose unit has three potential hydroxyl groups available for substitution, the maximum possible degree of substitution is 3. The degree of substitution is expressed as the number of moles of substituent groups per mole of anhydroglucose unit, on a molar average basis. The degree of substitution can be determined using proton nuclear magnetic resonance spectroscopy (“¹H NMR”) methods well known in the art. Suitable ¹H NMR techniques include those described in “Observation on NMR Spectra of Starches in Dimethyl Sulfoxide, Iodine-Complexing, and Solvating in Water-Dimethyl Sulfoxide”, Qin-Ji Peng and Arthur S. Perlin, Carbohydrate Research, 160 (1987), 57-72; and “An Approach to the Structural Analysis of Oligosaccharides by NMR Spectroscopy”, J. Howard Bradbury and J. Grant Collins, Carbohydrate Research, 71, (1979), 15-25.

The source of starch before chemical modification can be selected from a variety of sources such as tubers, legumes, cereal, and grains. For example, starch sources can include corn starch, wheat starch, rice starch, waxy corn starch, oat starch, cassaya starch, waxy barley, waxy rice starch, glutenous rice starch, sweet rice starch, amioca, potato starch, tapioca starch, oat starch, sago starch, sweet rice, or mixtures thereof. Suitable cationically modified starch polymers can be selected from degraded cationic maize starch, cationic tapioca, cationic potato starch, and mixtures thereof. Cationically modified starch polymers are cationic corn starch and cationic tapioca.

The starch, prior to degradation or after modification to a smaller molecular weight, can include one or more additional modifications. For example, these modifications may include cross-linking, stabilization reactions, phosphorylations, and hydrolyzations. Stabilization reactions can include alkylation and esterification.

Cationically modified starch polymers can be included in a cleansing composition in the form of hydrolyzed starch (e.g., acid, enzyme, or alkaline degradation), oxidized starch (e.g., peroxide, peracid, hypochlorite, alkaline, or any other oxidizing agent), physically/mechanically degraded starch (e.g., via the thermo-mechanical energy input of the processing equipment), or combinations thereof.

The starch can be readily soluble in water and can form a substantially translucent solution in water. The transparency of the composition is measured by Ultra-Violet/Visible (“UV/VIS”) spectrophotometry, which determines the absorption or transmission of UV/VIS light by a sample, using a Gretag Macbeth Colorimeter Color. A light wavelength of 600 nm has been shown to be adequate for characterizing the degree of clarity of cleansing compositions.

### Cationic Copolymer of an Acrylamide Monomer and a Cationic Monomer

A cleansing composition can include a cationic copolymer of an acrylamide monomer and a cationic monomer, wherein the copolymer has a charge density of 1.7 meq/g to 2.1 meq/g. The cationic copolymer can be a synthetic cationic copolymer of acrylamide monomers and cationic monomers.

Suitable cationic polymers can include:

(i) an acrylamide monomer of the following Formula IX:

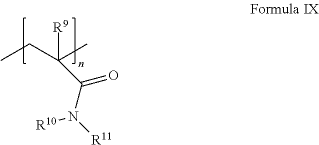

where R⁹ is H or C₁₋₄ alkyl; and R¹⁰ and R¹¹ are independently selected from the group consisting of H, C₁₋₄ alkyl, CH₂OCH₃, CH₂OCH₂CH(CH₃)₂, and phenyl, or together are C₃₋₆cycloalkyl; and

(ii) a cationic monomer conforming to Formula X:

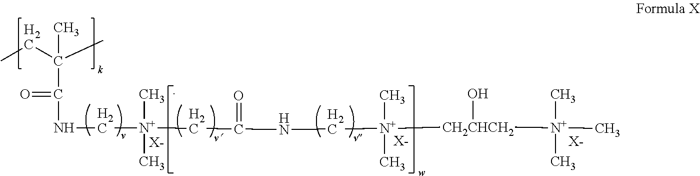

where k=1, each of v, v′, and v″ is independently an integer of from 1 to 6, w is zero or an integer of from 1 to 10, and X⁻ is an anion.

A cationic monomer can conform to Formula X where k=1, v=3 and w=0, z=1 and X⁻ is Cl⁻ to form the following structure (Formula XI):

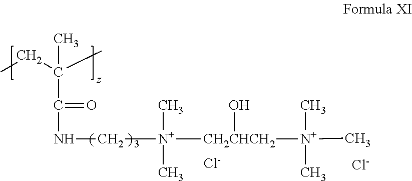

As can be appreciated, the above structure can be referred to as diquat.
A cationic monomer can conform to Formula X wherein v and v″ are each 3, v′=1, w=1, y=1 and X⁻ is Cl⁻, to form the following structure of Formula XII:

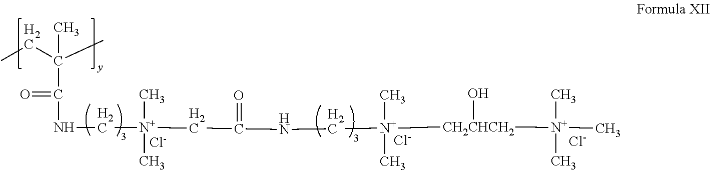

The structure of Formula XII can be referred to as triquat.

The acrylamide monomer can be either acrylamide or methacrylamide. The cationic copolymer can be AM:TRIQUAT which is a copolymer of acrylamide and 1,3-Propanediaminium,N-[2-[[[dimethyl[3-[(2-methyl-1-oxo-2-propenyl)amino]propyl]ammonio]acetyl]amino]ethyl]2-hydroxy-N,N,N′,N′ -pentamethyl-, trichloride. AM:TRIQUAT is also known as polyquaternium 76 (PQ76). AM:TRIQUAT can have a charge density of 1.6 meq/g and a M·Wt. of 1.1 million g/mol.

The cationic copolymer can include an acrylamide monomer and a cationic monomer, wherein the cationic monomer is selected from the group consisting of: dimethylaminoethyl (meth)acrylate, dimethylaminopropyl (meth)acrylate, ditertiobutylaminoethyl (meth)acrylate, dimethylaminomethyl (meth)acrylamide, dimethylaminopropyl (meth)acrylamide; ethylenimine, vinylamine, 2-vinylpyridine, 4-vinylpyridine; trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride, diallyldimethyl ammonium chloride, and mixtures thereof.

The cationic copolymer can include a cationic monomer selected from the group consisting of: trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride, and mixtures thereof.

The cationic copolymer can be formed from (1) copolymers of (meth)acrylamide and cationic monomers based on (meth)acrylamide, and/or hydrolysis-stable cationic monomers, (2) terpolymers of (meth)acrylamide, monomers based on cationic (meth)acrylic acid esters, and monomers based on (meth)acrylamide, and/or hydrolysis-stable cationic monomers. Monomers based on cationic (meth)acrylic acid esters can be cationized esters of the (meth)acrylic acid containing a quaternized N atom. Cationized esters of the (meth)acrylic acid containing a quaternized N atom can be quaternized dialkylaminoalkyl (meth)acrylates with C₁ to C₃ in the alkyl and alkylene groups. The cationized esters of the (meth)acrylic acid containing a quaternized N atom can be selected from the group consisting of ammonium salts of dimethylaminomethyl (meth)acrylate, dimethylaminoethyl (meth)acrylate, dimethylaminopropyl (meth)acrylate, diethylaminomethyl (meth)acrylate, diethylaminoethyl (meth)acrylate; and diethylaminopropyl (meth)acrylate quaternized with methyl chloride. The cationized esters of the (meth)acrylic acid containing a quaternized N atom can be dimethylaminoethyl acrylate, which is quaternized with an alkyl halide, or with methyl chloride or benzyl chloride or dimethyl sulfate (ADAME-Quat). The cationic monomer when based on (meth)acrylamides are quaternized dialkylaminoalkyl(meth)acrylamides with C₁ to C₃ in the alkyl and alkylene groups, or dimethylaminopropylacrylamide, which is quaternized with an alkyl halide, or methyl chloride or benzyl chloride or dimethyl sulfate.

The cationic monomer based on a (meth)acrylamide can be a quaternized dialkylaminoalkyl(meth)acrylamide with C₁ to C₃ in the alkyl and alkylene groups. The cationic monomer based on a (meth)acrylamide can be dimethylaminopropylacrylamide, which is quaternized with an alkyl halide, especially methyl chloride or benzyl chloride or dimethyl sulfate.

The cationic monomer can be a hydrolysis-stable cationic monomer. Hydrolysis-stable cationic monomers can be, in addition to a dialkylaminoalkyl(meth)acrylamide, any monomer that can be regarded as stable to the OECD hydrolysis test. The cationic monomer can be hydrolysis-stable, and the hydrolysis-stable cationic monomer can be selected from the group consisting of: diallyldimethylammonium chloride and water-soluble, cationic styrene derivatives.

The cationic copolymer can be a terpolymer of acrylamide, 2-dimethylammoniumethyl (meth)acrylate quaternized with methyl chloride (ADAME-Q) and 3-dimethylammoniumpropyl(meth)acrylamide quaternized with methyl chloride (DIMAPA-Q). The cationic copolymer can be formed from acrylamide and acrylamidopropyltrimethylammonium chloride, wherein the acrylamidopropyltrimethylammonium chloride has a charge density of 1.7 meq/g to 2.1 meq/g.

The cationic copolymer can have a charge density of 1.7 meq/g to 2.1 meq/g.

The cationic copolymer can have a M·Wt. 100 thousand g/mol to 2 million g/mol, 300 thousand g/mol to 1.8 million g/mol, 500 thousand g/mol to 1.6 million g/mol, 700 thousand g/mol to 1.4 million g/mol, and 900 thousand g/mol to 1.2 million g/mol.

The cationic copolymer can be AM:ATPAC. AM:ATPAC can have a charge density of 1.8 meq/g and a M·Wt. of 1.1 million g/mol.

### Synthetic Polymers

A cationic polymer can be a synthetic polymer that is formed from:
i) one or more cationic monomer units, and optionally
ii) one or more monomer units bearing a negative charge, and/or
iii) a nonionic monomer,
wherein the subsequent charge of the copolymer is positive. The ratio of the three types of monomers is given by “m”, “p” and “q” where “m” is the number of cationic monomers, “p” is the number of monomers bearing a negative charge and “q” is the number of nonionic monomers The cationic polymers can be water soluble or dispersible, non-crosslinked, and synthetic cationic polymers which have the structure of Formula XIII

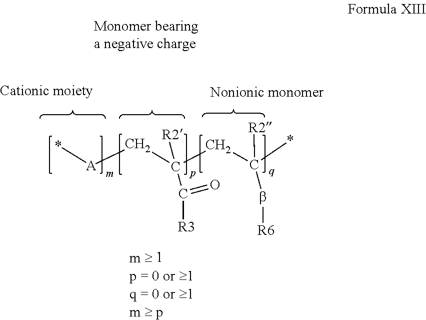

where A, may be one or more of the following cationic moieties:

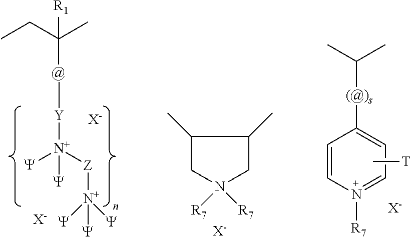

where @=amido, alkylamido, ester, ether, alkyl or alkylaryl;
where Y=C1-C22 alkyl, alkoxy, alkylidene, alkyl or aryloxy;
where Ψ=C1-C22 alkyl, alkyloxy, alkyl aryl or alkyl arylox;.
where Z=C1-C22 alkyl, alkyloxy, aryl or aryloxy;
where R1=H, C1-C4 linear or branched alkyl;
where s=0 or 1, n=0 or ≥1;
where T and R7=C1-C22 alkyl; and
where X−=halogen, hydroxide, alkoxide, sulfate or alkylsulfate.

Where the monomer bearing a negative charge is defined by R2′=H, C1-C4 linear or branched alkyl and R3 is:

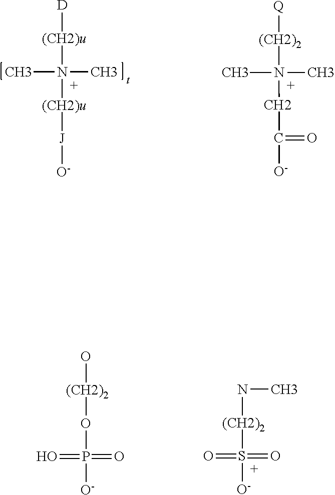

where D=O, N, or S;
where Q=NH₂ or O;
where u=1-6;
where t=0-1; and
where J=oxygenated functional group containing the following elements P, S, C.
   Where the nonionic monomer is defined by R2″=H, C₁-C₄ linear or branched alkyl, R6=linear or branched alkyl, alkyl aryl, aryl oxy, alkyloxy, alkylaryl oxy and β is defined as

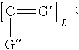

and
where G′ and G″ are, independently of one another, O, S or N—H and L=0 or 1.

Suitable monomers can include aminoalkyl (meth)acrylates, (meth)aminoalkyl (meth)acrylamides; monomers comprising at least one secondary, tertiary or quaternary amine function, or a heterocyclic group containing a nitrogen atom, vinylamine or ethylenimine; diallyldialkyl ammonium salts; their mixtures, their salts, and macromonomers deriving from therefrom.

Further examples of suitable cationic monomers can include dimethylaminoethyl (meth)acrylate, dimethylaminopropyl (meth)acrylate, ditertiobutylaminoethyl (meth)acrylate, dimethylaminomethyl (meth)acrylamide, dimethylaminopropyl (meth)acrylamide, ethylenimine, vinylamine, 2-vinylpyridine, 4-vinylpyridine, trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride, diallyldimethyl ammonium chloride.

Suitable cationic monomers can include quaternary monomers of formula —NR₃⁺, wherein each R can be identical or different, and can be a hydrogen atom, an alkyl group comprising 1 to 10 carbon atoms, or a benzyl group, optionally carrying a hydroxyl group, and including an anion (counter-ion). Examples of suitable anions include halides such as chlorides, bromides, sulphates, hydrosulphates, alkylsulphates (for example comprising 1 to 6 carbon atoms), phosphates, citrates, formates, and acetates.

Suitable cationic monomers can also include trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride. Additional suitable cationic monomers can include trimethyl ammonium propyl (meth)acrylamido chloride.

Examples of monomers bearing a negative charge include alpha ethylenically unsaturated monomers including a phosphate or phosphonate group, alpha ethylenically unsaturated monocarboxylic acids, monoalkylesters of alpha ethylenically unsaturated dicarboxylic acids, monoalkylamides of alpha ethylenically unsaturated dicarboxylic acids, alpha ethylenically unsaturated compounds comprising a sulphonic acid group, and salts of alpha ethylenically unsaturated compounds comprising a sulphonic acid group.

Suitable monomers with a negative charge can include acrylic acid, methacrylic acid, vinyl sulphonic acid, salts of vinyl sulfonic acid, vinylbenzene sulphonic acid, salts of vinylbenzene sulphonic acid, alpha-acrylamidomethylpropanesulphonic acid, salts of alpha-acrylamidomethylpropanesulphonic acid, 2-sulphoethyl methacrylate, salts of 2-sulphoethyl methacrylate, acrylamido-2-methylpropanesulphonic acid (AMPS), salts of acrylamido-2-methylpropanesulphonic acid, and styrenesulphonate (SS).

Examples of nonionic monomers can include vinyl acetate, amides of alpha ethylenically unsaturated carboxylic acids, esters of an alpha ethylenically unsaturated monocarboxylic acids with an hydrogenated or fluorinated alcohol, polyethylene oxide (meth)acrylate (i.e. polyethoxylated (meth)acrylic acid), monoalkylesters of alpha ethylenically unsaturated dicarboxylic acids, monoalkylamides of alpha ethylenically unsaturated dicarboxylic acids, vinyl nitriles, vinylamine amides, vinyl alcohol, vinyl pyrolidone, and vinyl aromatic compounds.

Suitable nonionic monomers can also include styrene, acrylamide, methacrylamide, acrylonitrile, methylacrylate, ethylacrylate, n-propylacrylate, n-butylacrylate, methylmethacrylate, ethylmethacrylate, n-propylmethacrylate, n-butylmethacrylate, 2-ethyl-hexyl acrylate, 2-ethyl-hexyl methacrylate, 2-hydroxyethylacrylate and 2-hydroxyethylmethacrylate.

The anionic counterion (X⁻) in association with the synthetic cationic polymers can be any known counterion so long as the polymers remain soluble or dispersible in water, in the cleansing composition, or in a coacervate phase of the cleansing composition, and so long as the counterions are physically and chemically compatible with the essential components of the cleansing composition or do not otherwise unduly impair product performance, stability or aesthetics. Non limiting examples of suitable counterions can include halides (e.g., chlorine, fluorine, bromine, iodine), sulfate, and methylsulfate.

The cationic polymer described herein can also aid in repairing damaged hair, particularly chemically treated hair by providing a surrogate hydrophobic F-layer. The microscopically thin F-layer provides natural weatherproofing, while helping to seal in moisture and prevent further damage. Chemical treatments damage the hair cuticle and strip away its protective F-layer. As the F-layer is stripped away, the hair becomes increasingly hydrophilic. It has been found that when lyotropic liquid crystals are applied to chemically treated hair, the hair becomes more hydrophobic and more virgin-like, in both look and feel. Without being limited to any theory, it is believed that the lyotropic liquid crystal complex creates a hydrophobic layer or film, which coats the hair fibers and protects the hair, much like the natural F-layer protects the hair. The hydrophobic layer can return the hair to a generally virgin-like, healthier state. Lyotropic liquid crystals are formed by combining the synthetic cationic polymers described herein with the aforementioned anionic detersive surfactant component of the cleansing composition. The synthetic cationic polymer has a relatively high charge density. It should be noted that some synthetic polymers having a relatively high cationic charge density do not form lyotropic liquid crystals, primarily due to their abnormal linear charge densities. Such synthetic cationic polymers are described in PCT Patent App. No. WO 94/06403 which is incorporated by reference. The synthetic polymers described herein can be formulated in a stable cleansing composition that provides improved conditioning performance, with respect to damaged hair.

Cationic synthetic polymers that provide enhanced conditioning and deposition of benefit agents can have a cationic charge density of 1.7 meq/g to 2.1 meq/g and can, but do not necessarily, form lytropic liquid crystals. The polymers also have a M·Wt. of 1,000 g/mol to 5,000,000 g/mol, 10,000 g/mol to 2,000,000 g/mol, and 100,000 g/mol to 2,000,000 g/mol.

### Cationic Cellulose Polymer

Suitable cationic polymers can be cellulose polymers. The cationic cellulose polymer can have a charge density 1.7 meq/g to 2.1 meq/g. Suitable cellulose polymers can include salts of hydroxyethyl cellulose reacted with trimethyl ammonium substituted epoxide, referred to in the industry (CTFA) as Polyquaternium 10 and available from Dwo/Amerchol Corp. (Edison, N.J., USA) in their Polymer KG series of polymers. Other suitable types of cationic cellulose can include the polymeric quaternary ammonium salts of hydroxyethyl cellulose reacted with lauryl dimethyl ammonium-substituted epoxide referred to in the industry (CTFA) as Polyquaternium 24. These materials are available from Dow/Amerchol Corp. under the tradename Polymer LM-200. Other suitable types of cationic cellulose can include the polymeric quaternary ammonium salts of hydroxyethyl cellulose reacted with lauryl dimethyl ammonium-substituted epoxide and trimethyl ammonium substituted epoxide referred to in the industry (CTFA) as Polyquaternium 67. These materials are available from Dow/Amerchol Corp. under the tradename SoftCAT Polymer SL-5, SoftCAT Polymer SL-30, Polymer SL-60, Polymer SL-100, Polymer SK-L, Polymer SK-M, Polymer SK-MH, and Polymer SK-H.

Additional cationic polymers are also described in the CTFA Cosmetic Ingredient Dictionary, 3rd edition, edited by Estrin, Crosley, and Haynes, (The Cosmetic, Toiletry, and Fragrance Association, Inc., Washington, D.C. (1982)), which is incorporated herein by reference.

Techniques for analysis of formation of complex coacervates are known in the art. For example, microscopic analyses of the compositions, at any chosen stage of dilution, can be utilized to identify whether a coacervate phase has formed. Such coacervate phase can be identifiable as an additional emulsified phase in the composition. The use of dyes can aid in distinguishing the coacervate phase from other insoluble phases dispersed in the composition. Additional details the use of cationic polymers and coacervates are disclosed in U.S. Pat. No. 9,272,164 which is incorporated by reference.

### Liquid Carrier

As can be appreciated, cleansing compositions can desirably be in the form of pourable liquid under ambient conditions. Inclusion of an appropriate quantity of a liquid carrier can facilitate the formation of a cleansing composition having an appropriate viscosity and rheology. A cleansing composition can include, by weight of the composition, 20% to 95%, by weight, of a liquid carrier, and 60% to 85%, by weight, of a liquid carrier. The liquid carrier can be an aqueous carrier such as water.

### Optional Components

As can be appreciated, cleansing compositions described herein can include a variety of optional components to tailor the properties and characteristics of the composition. As can be appreciated, suitable optional components are well known and can generally include any components which are physically and chemically compatible with the essential components of the cleansing compositions described herein. Optional components should not otherwise unduly impair product stability, aesthetics, or performance. Individual concentrations of optional components can generally range 0.001% to 10%, by weight of a cleansing composition. Optional components can be further limited to components which will not impair the clarity of a translucent cleansing composition.

Suitable optional components which can be included in a cleansing composition can include co-surfactants, deposition aids, conditioning agents (including hydrocarbon oils, fatty esters, silicones), anti-dandruff agents, suspending agents, viscosity modifiers, dyes, nonvolatile solvents or diluents (water soluble and insoluble), pearlescent aids, foam boosters, pediculocides, pH adjusting agents, perfumes, preservatives, chelants, proteins, skin active agents, sunscreens, UV absorbers, and vitamins The CTFA Cosmetic Ingredient Handbook, Tenth Edition (published by the Cosmetic, Toiletry, and Fragrance Association, Inc., Washington, D.C.) (2004) (hereinafter “CTFA”), describes a wide variety of non-limiting materials that can be added to the composition herein.

### Conditioning Agents

A cleansing composition can include a silicone conditioning agent. Suitable silicone conditioning agents can include volatile silicone, non-volatile silicone, or combinations thereof. If including a silicone conditioning agent, the agent can be included 0.01% to 10%, by weight of the composition, 0.1% to 8%, 0.1% to 5%, and/or 0.2% to 3%. Examples of suitable silicone conditioning agents, and optional suspending agents for the silicone, are described in U.S. Reissue Pat. No. 34,584, U.S. Pat. Nos. 5,104,646, and 5,106,609, each of which is incorporated by reference herein. Suitable silicone conditioning agents can have a viscosity, as measured at 25° C., 20 centistokes (“csk”) to 2,000,000 csk, 1,000 csk to 1,800,000 csk, 50,000 csk to 1,500,000 csk, and 100,000 csk to 1,500,000 csk.

The dispersed silicone conditioning agent particles can have a volume average particle diameter ranging 0.01 micrometer to 50 micrometer. For small particle application to hair, the volume average particle diameters can range 0.01 micrometer to 4 micrometer, 0.01 micrometer to 2 micrometer, 0.01 micrometer to 0.5 micrometer. For larger particle application to hair, the volume average particle diameters typically range 5 micrometer to 125 micrometer, 10 micrometer to 90 micrometer, 15 micrometer to 70 micrometer, and/or 20 micrometer to 50 micrometer.

Additional material on silicones including sections discussing silicone fluids, gums, and resins, as well as manufacture of silicones, are found in Encyclopedia of Polymer Science and Engineering, vol. 15, 2d ed., pp 204-308, John Wiley & Sons, Inc. (1989), which is incorporated herein by reference.

Silicone emulsions suitable for the cleansing compositions described herein can include emulsions of insoluble polysiloxanes prepared in accordance with the descriptions provided in U.S. Pat. No. 4,476,282 and U.S. Patent Application Publication No. 2007/0276087 each of which is incorporated herein by reference. Suitable insoluble polysiloxanes include polysiloxanes such as alpha, omega hydroxy-terminated polysiloxanes or alpha, omega alkoxy-terminated polysiloxanes having a molecular weight within the range 50,000 to 500,000 g/mol. The insoluble polysiloxane can have an average molecular weight within the range 50,000 to 500,000 g/mol. For example, the insoluble polysiloxane may have an average molecular weight within the range 60,000 to 400,000; 75,000 to 300,000; 100,000 to 200,000; or the average molecular weight may be 150,000 g/mol. The insoluble polysiloxane can have an average particle size within the range 30 nm to 10 micron. The average particle size may be within the range 40 nm to 5 micron, 50nm to 1 micron, 75 nm to 500 nm, or 100 nm, for example.

Other classes of silicones suitable for the cleansing compositions described herein can include i) silicone fluids, including silicone oils, which are flowable materials having viscosity less than 1,000,000 csk as measured at 25° C.; ii) aminosilicones, which contain at least one primary, secondary or tertiary amine; iii) cationic silicones, which contain at least one quaternary ammonium functional group; iv) silicone gums; which include materials having viscosity greater or equal to 1,000,000 csk as measured at 25° C.; v) silicone resins, which include highly cross-linked polymeric siloxane systems; vi) high refractive index silicones, having refractive index of at least 1.46, and vii) mixtures thereof.

Alternatively, the cleansing composition can be substantially free or free of silicones.

### Organic Conditioning Materials

The conditioning agent of the cleansing compositions described herein can also include at least one organic conditioning material such as oil or wax, either alone or in combination with other conditioning agents, such as the silicones described above. The organic material can be non-polymeric, oligomeric or polymeric. The organic material can be in the form of an oil or wax and can be added in the cleansing formulation neat or in a pre-emulsified form. Suitable examples of organic conditioning materials can include: i) hydrocarbon oils; ii) polyolefins, iii) fatty esters, iv) fluorinated conditioning compounds, v) fatty alcohols, vi) alkyl glucosides and alkyl glucoside derivatives; vii) quaternary ammonium compounds; viii) polyethylene glycols and polypropylene glycols having a molecular weight of up to 2,000,000 including those with CTFA names PEG-200, PEG-400, PEG-600, PEG-1000, PEG-2M, PEG-7M, PEG-14M, PEG-45M and mixtures thereof.

### Emulsifiers

A variety of anionic and nonionic emulsifiers can be used in the cleansing composition of the present invention. The anionic and nonionic emulsifiers can be either monomeric or polymeric in nature. Monomeric examples include, by way of illustrating and not limitation, alkyl ethoxylates, alkyl sulfates, soaps, and fatty esters and their derivatives. Polymeric examples include, by way of illustrating and not limitation, polyacrylates, polyethylene glycols, and block copolymers and their derivatives. Naturally occurring emulsifiers such as lanolins, lecithin and lignin and their derivatives are also non-limiting examples of useful emulsifiers.

### Chelating Agents

The cleansing composition can also comprise a chelant. Suitable chelants include those listed in A E Martell & R M Smith, Critical Stability Constants, Vol. 1, Plenum Press, New York & London (1974) and A E Martell & R D Hancock, Metal Complexes in Aqueous Solution, Plenum Press, New York & London (1996) both incorporated herein by reference. When related to chelants, the term “salts and derivatives thereof” means the salts and derivatives comprising the same functional structure (e.g., same chemical backbone) as the chelant they are referring to and that have similar or better chelating properties. This term includes alkali metal, alkaline earth, ammonium, substituted ammonium (i.e. monoethanolammonium, diethanolammonium, triethanolammonium) salts, esters of chelants having an acidic moiety and mixtures thereof, in particular all sodium, potassium or ammonium salts. The term “derivatives” also includes “chelating surfactant” compounds, such as those exemplified in U.S. Pat. No. 5,284,972, and large molecules comprising one or more chelating groups having the same functional structure as the parent chelants, such as polymeric EDDS (ethylenediaminedisuccinic acid) disclosed in U.S. Pat. Nos. 5,747,440. 5,284,972 and 5,747,440 are each incorporated by reference herein. Suitable chelants can further include histidine.

Levels of an EDDS chelant or histidine chelant in the cleansing compositions can be low. For example, an EDDS chelant or histidine chelant can be included at 0.01%, by weight. Above 10% by weight, formulation and/or human safety concerns can arise. The level of an EDDS chelant or histidine chelant can be at least 0.01%, by weight, at least 0.05%, by weight, at least 0.1%, by weight, at least 0.25%, by weight, at least 0.5%, by weight, at least 1%, by weight, or at least 2%, by weight, by weight of the cleansing composition.

### Gel Network

A cleansing composition can also include a fatty alcohol gel network. Gel networks are formed by combining fatty alcohols and surfactants in the ratio of 1:1 to 40:1, 2:1 to 20:1, and/or 3:1 to 10:1. The formation of a gel network involves heating a dispersion of the fatty alcohol in water with the surfactant to a temperature above the melting point of the fatty alcohol. During the mixing process, the fatty alcohol melts, allowing the surfactant to partition into the fatty alcohol droplets. The surfactant brings water along with it into the fatty alcohol. This changes the isotropic fatty alcohol drops into liquid crystalline phase drops. When the mixture is cooled below the chain melt temperature, the liquid crystal phase is converted into a solid crystalline gel network. Gel networks can provide a number of benefits to cleansing compositions. For example, a gel network can provide a stabilizing benefit to cosmetic creams and hair conditioners. In addition, gel networks can provide conditioned feel benefits to hair conditioners and shampoos.

A fatty alcohol can be included in the gel network at a level by weight of 0.05%, by weight, to 14%, by weight. For example, the fatty alcohol can be included in an amount ranging 1%, by weight, to 10%, by weight, and/or 6%, by weight, to 8%, by weight.

Suitable fatty alcohols include those having 10 to 40 carbon atoms, 12 to 22 carbon atoms, 16 to 22 carbon atoms, and/or 16 to 18 carbon atoms. These fatty alcohols can be straight or branched chain alcohols and can be saturated or unsaturated. Nonlimiting examples of fatty alcohols include cetyl alcohol, stearyl alcohol, behenyl alcohol, and mixtures thereof. Mixtures of cetyl and stearyl alcohol in a ratio of 20:80 to 80:20 are suitable.

A gel network can be prepared by charging a vessel with water. The water can then be heated to 74° C. Cetyl alcohol, stearyl alcohol, and surfactant can then be added to the heated water. After incorporation, the resulting mixture can passed through a heat exchanger where the mixture is cooled to 35° C. Upon cooling, the fatty alcohols and surfactant crystallized can form crystalline gel network. Table 1 provides the components and their respective amounts for an example gel network composition.

To prepare the gel network pre-mix of Table 1 Table 1, water is heated to 74° C. and the fatty alcohol and gel network surfactant are added to it in the quantities depicted in Table 1. After incorporation, this mixture is passed through a mill and heat exchanger where it is cooled to 32° C. As a result of this cooling step, the fatty alcohol, the gel network surfactant, and the water form a crystalline gel network.

TABLE 1

| Premix | % |
|---|---|
| Gel Network Surfactant¹ | 11.00 |
| Stearyl Alcohol | 8% |
| Cetyl Alcohol | 4% |
| Water | QS |

¹For amionic gel networks, suitable gel network surfactants above include surfactants with a net negative charge including sulfonates, carboxylates and phosphates among others and mixtures thereof.

For cationic gel networks, suitable gel network surfactants above include surfactants with a net positive charge including quaternary ammonium surfactants and mixtures thereof.

For Amphoteric or Zwitterionic gel networks, suitable gel network surfactants above include surfactants with both a positive and negative charge at product usage pH including betaines, amine oxides, sultaines, amino acids among others and mixtures thereof.

### Benefit Agents

A cleansing composition can further include one or more benefit agents. Exemplary benefit agents include, but are not limited to, particles, colorants, perfume microcapsules, gel networks, and other insoluble skin or hair conditioning agents such as skin silicones, natural oils such as sunflower oil or castor oil. The benefit agent can be selected from the group consisting of particles; colorants; perfume microcapsules; gel networks; other insoluble skin or hair conditioning agents such as skin silicones, natural oils such as sunflower oil or castor oil; and mixtures thereof.

### Suspending Agent

A cleansing composition can include a suspending agent at concentrations effective for suspending water-insoluble material in dispersed form in the compositions or for modifying the viscosity of the composition. Such concentrations range 0.1% to 10%, and 0.3% to 5.0%, by weight of the compositions. As can be appreciated however, suspending agents may not be necessary when certain glyceride ester crystals are included as certain glyceride ester crystals can act as suitable suspending or structuring agents.

Suitable suspending agents can include anionic polymers and nonionic polymers. Useful herein are vinyl polymers such as cross linked acrylic acid polymers with the CTFA name Carbomer, cellulose derivatives and modified cellulose polymers such as methyl cellulose, ethyl cellulose, hydroxyethyl cellulose, hydroxypropyl methyl cellulose, nitro cellulose, sodium cellulose sulfate, sodium carboxymethyl cellulose, crystalline cellulose, cellulose powder, polyvinylpyrrolidone, polyvinyl alcohol, guar gum, hydroxypropyl guar gum, xanthan gum, arabia gum, tragacanth, galactan, carob gum, guar gum, karaya gum, carragheenin, pectin, agar, quince seed (Cydonia oblonga Mill), starch (rice, corn, potato, wheat), algae colloids (algae extract), microbiological polymers such as dextran, succinoglucan, pulleran, starch-based polymers such as carboxymethyl starch, methylhydroxypropyl starch, alginic acid-based polymers such as sodium alginate, alginic acid propylene glycol esters, acrylate polymers such as sodium polyacrylate, polyethylacrylate, polyacrylamide, polyethyleneimine, and inorganic water soluble material such as bentonite, aluminum magnesium silicate, laponite, hectonite, and anhydrous silicic acid.

Other suitable suspending agents can include crystalline suspending agents which can be categorized as acyl derivatives, long chain amine oxides, and mixtures thereof. Examples of such suspending agents are described in U.S. Pat. No. 4,741,855, which is incorporated herein by reference. Suitable suspending agents include ethylene glycol esters of fatty acids having from 16 to 22 carbon atoms. The suspending agent can be an ethylene glycol stearate, both mono and distearate, can be acceptable, but particularly the distearate containing less than 7% of the mono stearate. Other suitable suspending agents include alkanol amides of fatty acids, having 16 to 22 carbon atoms, alternatively 16 to 18 carbon atoms, suitable examples of which include stearic monoethanolamide, stearic diethanolamide, stearic monoisopropanolamide and stearic monoethanolamide stearate. Other long chain acyl derivatives include long chain esters of long chain fatty acids (e.g., stearyl stearate, cetyl palmitate, etc.); long chain esters of long chain alkanol amides (e.g., stearamide diethanolamide distearate, stearamide monoethanolamide stearate); and glyceryl esters as previously described. Long chain acyl derivatives, ethylene glycol esters of long chain carboxylic acids, long chain amine oxides, and alkanol amides of long chain carboxylic acids can also be used as suspending agents.

Other long chain acyl derivatives suitable for use as suspending agents include N,N-dihydrocarbyl amido benzoic acid and soluble salts thereof (e.g., Na, K), particularly N,N-di(hydrogenated) C₁₆, C₁₈ and tallow amido benzoic acid species of this family, which are commercially available from Stepan® Company (Northfield, Ill., USA).

Examples of suitable long chain amine oxides for use as suspending agents include alkyl dimethyl amine oxides, e.g., stearyl dimethyl amine oxide.

Other suitable suspending agents include primary amines having a fatty alkyl moiety having at least 16 carbon atoms, examples of which include palmitamine or stearamine, and secondary amines having two fatty alkyl moieties each having at least 12 carbon atoms, examples of which include dipalmitoylamine or di(hydrogenated tallow) amine Still other suitable suspending agents include di(hydrogenated tallow)phthalic acid amide, and crosslinked maleic anhydride-methyl vinyl ether copolymer.

### Viscosity Modifiers

The shampoo composition can be free of or substantially free of viscosity modifiers other than organic salt.

In some examples, the composition can contain a viscosity modifier instead of or in addition to organic salt. Viscosity modifiers can be used to modify the rheology of a cleansing composition. Suitable viscosity modifiers can include Carbomers with tradenames Carbopol 934, Carbopol 940, Carbopol 950, Carbopol 980, and Carbopol 981, all available from B. F. Goodrich Company, acrylates/steareth-20 methacrylate copolymer with tradename ACRYSOL 22 available from Rohm and Hass, nonoxynyl hydroxyethylcellulose with tradename AMERCELL POLYMER HM-1500 available from Amerchol, methylcellulose with tradename BENECEL, hydroxyethyl cellulose with tradename NATROSOL, hydroxypropyl cellulose with tradename KLUCEL, cetyl hydroxyethyl cellulose with tradename POLYSURF 67, all supplied by Hercules, ethylene oxide and/or propylene oxide based polymers with tradenames CARBOWAX PEGs, POLYOX WASRs, and UCON FLUIDS, all supplied by Amerchol. Other suitable rheology modifiers can include cross-linked acrylates, cross-linked maleic anhydride co-methylvinylethers, hydrophobically modified associative polymers, and mixtures thereof.

### Dispersed Particles

Dispersed particles as known in the art can be included in a cleansing composition. If including such dispersed particles, the particles can be incorporated, by weight of the composition, at levels of 0.025% or more, 0.05% or more, 0.1% or more, 0.25% or more, and 0.5% or more. However, the cleansing compositions can also contain, by weight of the composition, 20% or fewer dispersed particles, 10% or fewer dispersed particles, 5% or fewer dispersed particles, 3% or fewer dispersed particles, and 2% or fewer dispersed particles.

As can be appreciated, a cleansing composition can include still further optional components. For example, amino acids can be included. Suitable amino acids can include water soluble vitamins such as vitamins B1, B2, B6, B12, C, pantothenic acid, pantothenyl ethyl ether, panthenol, biotin, and their derivatives, water soluble amino acids such as asparagine, alanin, indole, glutamic acid and their salts, water insoluble vitamins such as vitamin A, D, E, and their derivatives, water insoluble amino acids such as tyrosine, tryptamine, and their salts.

Anti-dandruff agents can be included. As can be appreciated, the formation of a coacervate can facilitate deposition of the anti-dandruff agent to the scalp.

A cleansing composition can optionally include pigment materials such as inorganic, nitroso, monoazo, disazo, carotenoid, triphenyl methane, triaryl methane, xanthene, quinoline, oxazine, azine, anthraquinone, indigoid, thionindigoid, quinacridone, phthalocianine, botanical, natural colors, including: water soluble components such as those having C. I. Names.

The compositions can also include antimicrobial agents which are useful as cosmetic biocides and antidandruff agents including: pyridinethione salts, zinc pyrithione, azoles, selenium sulfide, particulate sulfur, coal tar, sulfur, whitfield's ointment, castellani's paint, aluminum chloride, gentian violet, piroctone olamine, ciclopirox olamine, undecylenic acid and it's metal salts, potassium permanganate, selenium sulphide, sodium thiosulfate, propylene glycol, oil of bitter orange, urea preparations, griseofulvin, 8-hydroxyquinoline ciloquinol, thiobendazole, thiocarbamates, haloprogin, polyenes, hydroxypyridone, morpholine, benzylamine, allylamines (such as terbinafine), tea tree oil, clove leaf oil, coriander, palmarosa, berberine, thyme red, cinnamon oil, cinnamic aldehyde, citronellic acid, hinokitol, ichthyol pale, Sensiva SC-50, Elestab HP-100, azelaic acid, lyticase, iodopropynyl butylcarbamate (IPBC), isothiazalinones such as octyl isothiazalinone and azoles, azoxystrobin and combinations thereof.

One or more stabilizers and preservatives can be included. For example, one or more of trihydroxystearin, ethylene glycol distearate, citric acid, sodium citrate dihydrate, a preservative such as kathon, sodium chloride, sodium benzoate, sodium salicylate and ethylenediaminetetraacetic acid (“EDTA”) can be included to improve the lifespan of a personal care compositon. The stabilizer and/or preservative can be used at a level of 0.10 wt % to 2 wt %. Particularly suitable is sodium benzoate at a level of 0.10 wt % to 0.45 wt %. The personal care composition may also include citric acid at a level of 0.5 wt % to 2 wt %. The sodium benzoate and the citric acid can be added to the personal care composition alone or in combination.

### Method of Making a Cleansing Composition

A cleansing composition described herein can be formed similarly to known cleansing compositions. For example, the process of making a cleansing composition can include the step of mixing the surfactant, cationic polymer, and liquid carrier together to form a cleansing composition.

Additional information on sulfate-free surfactants and other ingredients that are suitable for shampoo compositions is found at U.S. Pub. Nos. 2019/0105247 and 2019/0105246, incorporated by reference.

### Methods

### Argentometry Method to Measure wt % Inorganic Chloride Salts

The weight % of inorganic chloride salt in the composition can be measured using a potentiometric method where the chloride ions in the composition are titrated with silver nitrate. The silver ions react with the chloride ions from the composition to form an insoluble precipitate, silver chloride. The method used an electrode (Mettler Toledeo DM141) that is designed for potentiometric titrations of anions that precipitate with silver. The largest change in the signal occurs at the equivalence point when the amount of added silver ions is equal to the amount of chloride ions in solution. The concentration of silver nitrate solution used should be calibrated using a chloride solution known to one of skill in the art, such as a sodium chloride solution that contains a standard and known amount of sodium chloride to confirm that the results match the known concentration. This type of titration involving a silver ion is known as argentometry and is commonly used to determine the amount of chloride present in a sample.

### Methods to Determine Lack of In Situ Coacervate in Composition Prior to Dilution

### 1. Microscopy Method to Determine Lack of In Situ Coacervate

Lack of in situ coacervate can be determined using a microscope. The composition is mixed to homogenize, if needed. Then, the composition is sampled onto a microscope slide and mounted on a microscope, per typical microscopy practices. The sample is viewed at, for example, a 10× or 20× objective. If in situ coacervate is present in the sample, an amorphous, gel-like phase with 20 nm to 200 nm particle size can be seen throughout the sample. This amorphous, gel-like phases can be described as gel chunks or globs. In this method, the in situ coacervate is separate from other ingredients that were intentionally added to the formula that form flocks or otherwise appear as particles under microscopy.

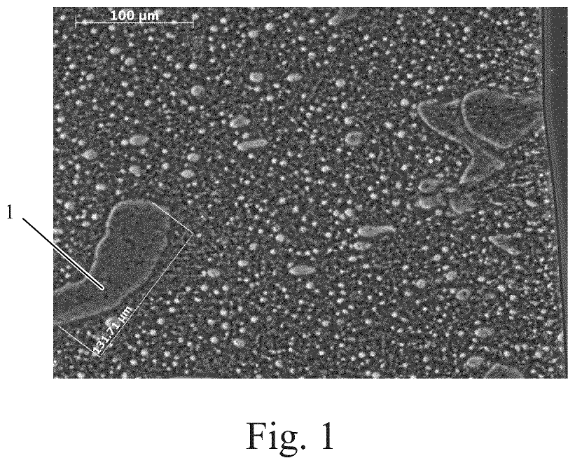

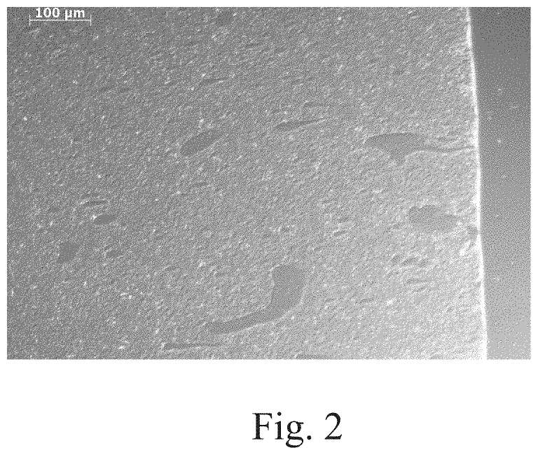

FIG. 1 is an example microscopy photograph at 20× objective of a marketed sulfate-free shampoo composition that contains a cationic polymer and also has in situ coacervate. FIG. 1 at reference numeral 1 shows an amorphous, gel-like phase that is 130 nm long that is the in situ coacervate. FIG. 2 is an example microscopy photograph at 10× objective the same marketed shampoo composition that was used in FIG. 1 at 20× objective. FIG. 2 shows many of these amorphous, gel-like phases present with a length 20 nm to 200 nm.

### 2. Clarity Assessment—Measurement of % Transmittance (% T)

Lack of in situ coacervate can be determined by composition clarity. A composition that does not contain in situ coacervate will be clear, if it does not contain any ingredients that would otherwise give it a hazy appearance.

Composition clarity can be measured by % Transmittance. For this assessment to determine if the composition lacks coacervate, the composition should be made without ingredients that would give the composition a hazy appearance such as silicones, opacifiers, non-silicone oils, micas, and gums or anionic rheology modifiers. It is believed that adding these ingredients would not cause in situ coacervate to form prior to use, however these ingredients will obscure measurement of clarity by % Transmittance.

Clarity can be measured by % Transmittance (% T) using Ultra-Violet/Visible (UV/VI) spectrophotometry which determines the transmission of UV/VIS light through a sample. A light wavelength of 600 nm has been shown to be adequate for characterizing the degree of light transmittance through a sample. Typically, it is best to follow the specific instructions relating to the specific spectrophotometer being used. In general, the procedure for measuring percent transmittance starts by setting the spectrophotometer to 600 nm. Then a calibration “blank” is run to calibrate the readout to 100 percent transmittance. A single test sample is then placed in a cuvette designed to fit the specific spectrophotometer and care is taken to ensure no air bubbles are within the sample before the % T is measured by the spectrophotometer at 600 nm. Alternatively, multiple samples can be measured simultaneously by using a spectrophotometer such as the SpectraMax M-5 available from Molecular Devices. Multiple samples are transferred into a 96 well visible flat bottom plate (Greiner part #655-001), ensuring that no air bubbles are within the samples. The flat bottom plate is placed within the SpectraMax M-5 and % T measured using the Software Pro v.5™ software available from Molecular Devices.

### 3. Lasentec FBRM Method

Lack of in situ coacervate can also be measured using Lasentec FBRM Method with no dilution. A Lasentec Focused Beam Reflectance Method (FBRM) [model S400A available from Mettler Toledo Corp] may be used to determine floc size and amount as measured by chord length and particle counts/sec (counts per sec). A composition that is free of flocs can lack in situ coacervate. A composition can have flocs and also be free of in situ coacervate if the flocs are known to be the added particles.

### 4. In Situ Coacervate Centrifuge Method

Lack of in situ coacervate can also be measured by centrifuging a composition and measuring in situ coacervate gravimetrically. For this method, the composition should be made without a suspending agent to allow for separation of an in situ coacervate phase. The composition is centrifuged for 20 minutes at 9200 rpm using a Beckman Couller TJ25 centrifuge. Several time/rpm combinations can be used. The supernatant is then removed and the remaining settled in situ coacervate assessed gravimetrically. % In Situ Coacervate is calculated as the weight of settled in situ coacervate as a percentage of the weight of composition added to the centrifuge tube using the equation below. This quantifies the percentage of the composition that participates in the in situ coacervate phase.

$$\% In Situ Coacervate = \frac{Weight of settled in situ coacervate}{Weight of composition added to centrifuge tube} \times 100$$

### Measures of Improved Performance Due to No In Situ Coacervate Prior to Dilution

The composition does not contain in situ coacervate prior to dilution. Because of this, coacervate quantity and quality upon dilution is better than a composition that does contain in situ coacervate prior to dilution. This provides better wet conditioning and deposition of actives from a composition that does not contain coacervate prior to dilution compared to a composition that does contain coacervate prior to dilution.

### 1. Measurement of % Transmittance (% T) During Dilution

Coacervate formation upon dilution for a transparent or translucent composition can be assessed using a spectrophotometer to measure the percentage of light transmitted through the diluted sample (% T). As percent light transmittance (% T) values measured of the dilution decrease, typically higher levels of coacervate are formed. Dilutio ns samples at various weight ratios of water to composition can be prepared, for example 2 parts of water to 1 part composition (2:1), or 7.5 parts of water to 1 part composition (7.5:1), or 16 parts of water to 1 part composition (16:1), or 34 parts of water to 1 part composition (34:1), and the % T measured for each dilution ratio sample. Examples of possible dilution ratios may include 2:1, 3:1, 5:1, 7.5:1, 11:1, 16:1, 24:1, or 34:1. By averaging the % T values for samples that span a range of dilution ratios, it is possible to simulate and ascertain how much coacervate a composition on average would form as a consumer applies the composition to wet hair, lathers, and then rinses it out. Average % T can be calculated by taking the numerical average of individual % T measurements for the following dilution ratios: 2:1, 3:1, 5:1, 7.5:1, 11:1, 16:1, 24:1, and 34:1. Lower average %T indicates more coacervate is formed on average as a consumer applies the composition to wet hair, lathers and then rinses it out.

% T can be measured using Ultra-Violet/Visible (UV/VI) spectrophotometry which determines the transmission of UV/VIS light through a sample. A light wavelength of 600 nm has been shown to be adequate for characterizing the degree of light transmittance through a sample. Typically, it is best to follow the specific instructions relating to the specific spectrophotometer being used. In general, the procedure for measuring percent transmittance starts by setting the spectrophotometer to 600 nm. Then a calibration “blank” is run to calibrate the readout to 100 percent transmittance. A single test sample is then placed in a cuvette designed to fit the specific spectrophotometer and care is taken to insure no air bubbles are within the sample before the % T is measured by the spectrophotometer at 600 nm. Alternatively, multiple samples can be measured simultaneously by using a spectrophotometer such as the SpectraMax M-5 available from Molecular Devices. Multiple dilution samples can be prepared within a 96 well plate (VWR catalog#82006-448) and then transferred to a 96 well visible flat bottom plate (Greiner part #655-001), ensuring that no air bubbles are within the sample. The flat bottom plate is placed within the SpectraMax M-5 and % T measured using the Software Pro v.5™ software available from Molecular Devices.

### 2. Assessment of Coacervate Floc Size Upon Dilution

Coacervate floc size upon dilution can be assessed visually. Dilutions samples at various weight ratios of water to composition can be prepared, for example 2 parts of water to 1 part composition (2:1), or 7.5 parts of water to 1 part composition (7.5:1), or 16 parts of water to 1 part composition (16:1), or 34 parts of water to 1 part composition (34:1), and the % T measured for each dilution ratio sample. Examples of possible dilution ratios may include 2:1, 3:1, 5:1, 7.5:1, 11:1, 16:1, 24:1, or 34:1. Larger coacervate flocs can indicate a better quality coacervate that provides better wet conditioning and deposition of actives.

### 3. Wet Combing Force Method

Hair switches of 4 grams general population hair at 8 inches length are used for the measurement. Each hair switch is treated with 4 cycles (1 lather/rinse steps per cycle, 0.1 gm cleansing composition/gm hair on each lather/rinse step, drying between each cycle) with the cleansing composition. Four switches are treated with each shampoo. The hair is not dried after the last treatment cycle. While the hair is wet, the hair is pulled through the fine tooth half of two Beautician 3000 combs. Force to pull the hair switch through the combs is measured by a friction analyzer (such as Instron or MTS tensile measurement) with a load cell and outputted in gram-force (gf). The pull is repeated for a total of five pulls per switch. Average wet combing force is calculated by averaging the force measurement from the five pulls across the four hair switches treated with each cleansing composition. Data can be shown as average wet combing force through one or both of the two combs.

### 4. Deposition Method

Deposition of actives can be measured in vitro on hair tresses or in vivo on panelist's heads. The composition is dosed on a hair tress or panelist head at a controlled amount and washed according to a conventional washing protocol. For a hair tress, the tress can be sampled and tested by an appropriate analytical measure to determine quantity deposited of a given active. To measure deposition on a panelist's scalp, the hair is then parted on an area of the scalp to allow an open-ended glass cylinder to be held on the surface while an aliquot of an extraction solution is added and agitated prior to recovery and analytical determination of a given active. To measure deposition on a panelist's hair, a given amount of hair is sampled and then tested by an appropriate analytical measure to determine quantity deposited of a given active.

### Cone/Plate Viscosity Measurement

The viscosities of the examples are measured by a Cone/Plate Controlled Stress Brookfield Rheometer R/S Plus, by Brookfield Engineering Laboratories, Stoughton, Mass. The cone used (Spindle C-75-1) has a diameter of 75 mm and 1° angle. The liquid viscosity is determined using a steady state flow experiment at constant shear rate of 2 s⁻¹ and at temperature of 26.7° C. The sample size is 2.5 ml to 3 ml and the total measurement reading time is 3 minutes.

### Lather Characterization—Kruss DFA100 Lather Characterization

A cleansing composition dilution of 10 parts by weight water to 1 part by weight cleanser is prepared. The shampoo dilution is dispensed into the Kruss DFA100 which generates the lather and measures lather properties.

### pH Method

First, calibrate the Mettler Toledo Seven Compact pH meter. Do this by turning on the pH meter and waiting for 30 seconds. Then take the electrode out of the storage solution, rinse the electrode with distilled water, and carefully wipe the electrode with a scientific cleaning wipe, such as a Kimwipe®. Submerse the electrode in the pH 4 buffer and press the calibrate button. Wait until the pH icon stops flashing and press the calibrate button a second time. Rinse the electrode with distilled water and carefully wipe the electrode with a scientific cleaning wipe. Then submerse the electrode into the pH 7 buffer and press the calibrate button a second time. Wait until the pH icon stops flashing and press the calibrate button a third time. Rinse the electrode with distilled water and carefully wipe the electrode with a scientific cleaning wipe. Then submerse the electrode into the pH 10 buffer and press the calibrate button a third time. Wait until the pH icon stops flashing and press the measure button. Rinse the electrode with distilled water and carefully wipe with a scientific cleaning wipe. Submerse the electrode into the testing sample and press the read button. Wait until the pH icon stops flashing and record the value.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention.

The following Examples illustrate various shampoo compositions. Each composition was prepared by conventional formulation and mixing techniques.

The total sodium chloride in the tables below was calculated based on the product specifications from the suppliers. Some of the surfactants used in the examples below are sourced in a liquid mixture containing the surfactant at some active concentration, water, and often sodium chloride at some level generated during synthesis of the surfactant. For example, a common surfactant synthesis that produces sodium chloride as a byproduct is the synthesis of cocamidopropyl betaine. In this synthesis, an amidoamine is reacted with sodium monochloroacetate to produce betaine and sodium chloride. This is one example of a surfactant synthesis that produces sodium chloride as a byproduct. Public supplier documents including example Certificate of Analysis and Technical Specification documents list activity by wt % or solids by wt % and wt % sodium chloride. Using these specifications and the surfactant activity in the composition, inherent levels of sodium chloride coming in with the surfactants can be summed up for a given composition and added to any sodium chloride that is directly added to the composition. While surfactants are a common raw material that introduces sodium chloride into the formula, other materials can also be checked for content of sodium chloride to include in the overall sodium chloride calculation. For calculation of total inorganic salt, this total sodium chloride is added to any other inorganic salts that are added through a raw material or intentionally. The ratio of anionic surfactant to amphoteric surfactant is calculated by wt. %.

Shampoo compositions with surfactant systems that are substantially free of sulfate-based surfactants can have low viscosity, which makes it more difficult to apply across a user's hair and scalp without it running through their fingers. Example 1 was made, and it was determined that Example 1 had consumer acceptable viscosity and therefore, Example 1 serves as the reference for other examples. The viscosity of the other examples was compared to Example 1 and was considered consumer acceptable if by visual inspection it appeared to have a viscosity approximately equal to or greater than the viscosity of Example 1. The viscosity was not consumer acceptable if by visual inspection it appeared to have a viscosity less than that of Example 1. The visual inspection was performed as follows: after the sample was made, it was put into a transparent container and gently rocked and the flow of the liquid was observed by a person with an unaided eye (excepting standard corrective lenses adapted to compensate for near-sightedness, farsightedness, or stigmatism, or other corrected vision) in lighting at least equal to the illumination of a standard 100 watt incandescent white light bulb at a distance of 20 cm. All examples were made at similar pH.

If the example appeared to have a viscosity that was approximately greater than or equal to the viscosity of Example 1, then it was presumed that the micelle elongation was sufficient. The micelle elongation was presumed to be insufficient if the formula appeared to have a viscosity less than Example 1.

For the examples and comparative examples in Table 2 and Table 3, the in situ coacervate was determined as follows. The examples were prepared as described herein. The example was made and immediately put in a clear, glass jar of at least 1 inch width. The cap was screwed on the jar, finger tight. The example was stored at ambient temperatures (20-25° C.), away from direct sunlight, for 5 days. For some examples, the composition was stored for up to 9 months to determine if there was phase separation. Then the composition was inspected to see if either haze or precipitate was visually detectable. If either haze or precipitate were present, it was determined that the composition had in situ coacervate. If neither haze nor precipitate were present, it was determined that there was no in situ coacervate. It is believed that the shampoo product would have improved conditioning performance as compared to examples where in situ coacervate formed.

The example was inspected to determine if haze could be visually detected. If the example was clear, then there was no in situ coacervate and it is believed that the shampoo product would have improved conditioning performance as compared to examples where in situ coacervate formed. If haze was detected in the example, then there was in situ coacervate and it is believed that the example would be less preferred by consumers.

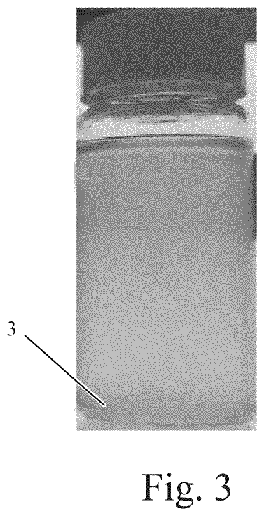

The example was also inspected to determine a separated phase formed on the bottom of the jar. This phase will form in as short as 3 days, but could take up to 9 months depending on the viscosity of the composition. FIG. 3 is a photograph of Comparative Example 4 (C4) after 9 months of storage. Reference numeral 3 is a separated coacervate phase at the bottom of the jar. As used herein, “visually detect” or “visually detectable” means that a human viewer can visually discern the quality of the example with the unaided eye (excepting standard corrective lenses adapted to compensate for near-sightedness, farsightedness, or stigmatism, or other corrected vision) in lighting at least equal to the illumination of a standard 100 watt incandescent white light bulb at a distance of 1 meter.

The examples in Table 2 Table 2 and Table 3Table 3 could also be formulated with silicones, opacifiers (e.g. glycol distearate, glycol stearate), non-silicone oils, micas, gums or anionic rheology modifiers and other ingredients that would cause the shampoo to have a hazy appearance. However, it is believed that adding these ingredients would not cause in situ coacervate to form prior to use.

TABLE 2

| | Ex.1 | C1 | C2 | C3 | C4 |
|---|---|---|---|---|---|
| | (wt. %) | (wt. %) | (wt. %) | (wt. %) | (wt. %) |
| Lauramidopropyl | 2.44 | 5.36 | 9.75 | 9.75 | — |
| Betaine ¹ | | | | | |
| Cocamidopropyl | 7.31 | 4.39 | — | — | 9.75 |
| Betaine ¹⁰ | | | | | |
| Total Amphoteric | 9.75 | 9.75 | 9.75 | 9.75 | 9.75 |
| Surfactant | | | | | |
| Sodium Cocoyl | 6.00 | 6.00 | 6.00 | 6.00 | 6.00 |
| Isethionate ² | | | | | |
| Sodium Lauroyl | 2.5 | 2.5 | 2.5 | 2.5 | 2.5 |
| Sarcosinate ³ | | | | | |
| Polyquaternium- | 0.4 | — | — | 0.55 | 0.55 |
| 10 ⁴ (KG-30M, CD 1.9 | | | | | |
| meq/g) | | | | | |
| Polyquaternium- | — | 0.4 | 0.55 | — | — |
| 10 ⁵ (JR-30M, CD | | | | | |
| 1.25 meq/g) | | | | | |
| Sodium Benzoate ⁸ | 0.75 | 0.75 | 0.75 | 0.75 | 0.75 |
| Sodium Salicylate ⁹ | 0.45 | 0.45 | 0.45 | 0.45 | 0.45 |
| Tetrasodium EDTA ¹¹ | 0.16 | 0.16 | 0.16 | 0.16 | 0.16 |
| Silicone ¹² | 0.25 | 0.25 | — | — | 0.95 |
| Perfume | 1.2 | 1.2 | 1.1 | 1.1 | 1.2 |
| Added Sodium | 0 | 0.3 | 0 | 0 | 0.3 |
| Chloride ⁶ | | | | | |
| Citric Acid | To pH 5.5 to 6.0 | | | | |
| Water and Optional | Q.S. to 100 | | | | |
| Components | | | | | |
| Ratio of Anionic to | 0.9:1 | 0.9:1 | 0.9:1 | 0.9:1 | 0.9:1 |
| Amphoteric Surfactant | | | | | |
| Total Sodium Chloride | 1.3 | 1.1 | 0.07 | 0.07 | 2.0 |
| (including from | | | | | |
| surfactant) | | | | | |
| Viscosity | Reference | ≥Reference | <Reference | <Reference | ≥Reference |
| Micelle Elongation | Sufficient | Sufficient | Insufficient | Insufficient | Sufficient |
| Contains in situ | No | Yes | No | No | Yes |
| coacervate prior to | | | | | |
| Dilution? | | | | | |

Example 1 contained polyquaternium-10 with a charge density of 1.9 meq/g and 1.3% sodium chloride and had sufficient viscosity and was clear and there was no observed phase separation, which indicated that there was no in situ coacervate. It is believed that Example 1 would have good conditioning performance and be preferred by consumers, as compared to Comparative Examples 1-4. Comparative Examples 1 (C1) and 4 (C4) were hazy and/or had a separate phase that was present at the bottom of the jar, indicating the presence of in situ coacervate. Comparative Examples 2 and 3 had a viscosity that was lower than Example 1 and therefore may not be consumer preferred.

Comparative Example 1 (C1) had polyquaternium-10 with a charge density of 1.25 meq/g and 1.1% sodium chloride. C1 was not stable because it was hazy and/or had a separate phase that was present at the bottom of the jar, indicating the presence of in situ coacervate. C1 is believed to have less conditioning performance and will not be consumer preferred. Comparative Example 2 (C2) also had polyquaternium-10 with a charge density of 1.25 meq/g and the viscosity of this formulas was insufficient. As shown in C1 and C2, sulfate-free surfactant systems with cationic polymers with a lower charge density (e.g. 1.25 meq/g), may not form compositions that are consumer preferred.

Comparative Example 3 (C3) had polyquaternium-10 with a charge density of 1.9 meq/g and 0.1% sodium chloride. The viscosity of C3 was insufficient. Comparative Example 4 (C4) had polyquaternium-10 with a charge density of 1.9 meq/g and 2% sodium chloride. C4 was not stable because it had a separate phase that was present at the bottom of the jar (see FIG. 3), indicating the presence of in situ coacervate. C4 is believed to have less conditioning performance and will not be consumer preferred. As shown in C3 and C4, sulfate-free surfactant systems with too much (e.g., 2% total inorganic salt) or too little (e.g., 0.1% total inorganic salt), may not form compositions that are consumer preferred.

TABLE 3
Shampoo Compositions

| | Ex. 2 | Ex. 3 | Ex. 4 | Ex. 5 | Ex. 6 | Ex. 7 | Ex. 8 |
|---|---|---|---|---|---|---|---|
| | (wt. %) | (wt. %) | (wt. %) | (wt. %) | (wt. %) | (wt. %) | (wt. %) |
| Lauramidopropyl Betaine ¹ | 2.44 | 2.44 | — | 2.44 | 2.44 | 2.44 | — |
| Low Salt Cocamidopropyl Betaine ⁷ | — | — | 9.75 | — | — | — | — |
| Cocamidopropyl Betaine ¹⁰ | 7.31 | 7.31 | — | 7.31 | 7.31 | 7.31 | 7.5 |
| Total Amphoteric Surfactant | 9.75 | 9.75 | 9.75 | 9.75 | 9.75 | 9.75 | 7.5 |
| Sodium Cocoyl Isethionate ² | 6.00 | 6.00 | 6.00 | 6.00 | 6.00 | 6.00 | 4.5 |
| Sodium Lauroyl Sarcosinate ³ | 4 | — | 2.5 | 2.5 | 2.5 | 2.5 | — |
| Polyquatemium-10 ⁴ | 0.4 | 0.4 | 0.4 | 0.05 | 0.55 | 0.15 | 0.25 |
| (KG-30M, CD 1.9 meq/g) | | | | | | | |
| Piroctone Olamine ¹³ | — | — | — | — | — | 0.5 | — |
| Zinc Pyrithione ¹⁴ | — | — | — | — | — | — | 1 |
| Acrylates Copolymer ¹⁵ | — | — | — | — | — | — | 0.7 |
| Added Sodium Chloride ⁶ | 0 | 0 | 1.2 | 0 | 0.2 | 0 | 0 |
| Water, Preservatives, | Q.S. to 100 | | | | | | |
| pH adjusters, Fragrance | | | | | | | |
| and Optional Components | | | | | | | |
| Ratio of Anionic to | 1.0:1 | 0.6:1 | 0.9:1 | 0.9:1 | 0.9:1 | 0.9:1 | 0.6:1 |
| Amphoteric Surfactant | | | | | | | |
| Total Sodium Chloride | 1.3 | 1.3 | 1.3 | 1.3 | 1.5 | 1.3 | 1.3 |
| (including from surfactant) | | | | | | | |

Examples 3 and 5-8 were made and contained polyquaternium-10 with a charge density of 1.9 meq/g and 1.3-1.5% sodium chloride and had sufficient viscosity. Examples 3 and 5-7 were clear and there was no observed phase separation, which indicated that there was no in situ coacervate. Example 8 is opaque, however, there was no observed phase separation and therefore it was presumed that there was no in situ coacervate. It is believed that Examples 3 and 5-8 would have good conditioning performance and be preferred by consumers.

Examples 2 and 4 could be made. It is expected that these formulas would have sufficient viscosity and micelle elongation and no in situ coacervate would form prior to dilution. It is believed that Examples 2 and 4 would also be consumer preferred.

TABLE 4

| | Ex. 9 | Ex. 10 |
|---|---|---|
| | (wt. %) | (wt. %) |
| Lauramidopropyl Betaine ¹ | — | 2.44 |
| Cocamidopropyl Betaine ¹⁰ | 7.5 | 7.31 |
| Sodium Cocoyl Isethionate ² | 4.5 | 6 |
| Sodium Lauroyl Sarcosinate ³ | — | 2.5 |
| Polyquaternium-10 ⁴ | 0.4 | 0.4 |
| (KG-30M, CD 1.9 meq/g) | | |
| Sodium Benzoate ⁸ | 0.75 | 0.45 |
| Sodium Salicylate ⁹ | 0.45 | 0.45 |
| Perfume | 1 | 1 |
| Citric Acid | to pH 5.5-6.5 | to pH 5.5-6.5 |
| Water | Q.S. to 100 | Q.S. to 100 |
| Ratio of Anionic to | 0.6:1 | 0.9:1 |
| Amphoteric Surfactant | | |
| Total Sodium Chloride | 1.3 | 1.3 |
| (including from surfactant) | | |

Examples 9 and 10 could be made. It is expected that these formulas would have sufficient viscosity and micelle elongation and no in situ coacervate would form prior to dilution. It is believed that Examples 10 and 11 would also be consumer preferred.

### Suppliers for Examples in Table 2Tables 2, 3, and 4:

Mackam DAB-ULS available from Solvay. Specification Range: Solids=34-36%, Sodium Chloride=0-0.5%. Average values are used for calculations: Actives=35%, Sodium Chloride=0.25%.
2. Hostapon SCI-85 C available from Clariant (0% Sodium Chloride)
3. SP Crodasinic LS30/NP MBAL available from Croda (<0.2% Sodium Chloride)
4. UCARE Polymer KG-30M available from Dow
5. UCARE Polymer JR-30M available from Dow
6. Sodium Chloride available from Norton International Inc.
7. Dehyton PK 45 from BASF with Sodium Chloride removed, resulting in 33.05% Dry Residue, 0.21% Sodium Chloride
8. Sodium Benzoate available from Kalama Chemical
9. Sodium Salicylate available from JQC (Huayin) Pharmaceutical Co., Ltd
10. Tego Betain CK PH 12 available from Evonik. Specification Range: Actives=28-32%, Sodium Chloride=4.5-6%. Average values are used for calculations: Actives=30%, Sodium Chloride=5.25%.
11. Versene 220 Crystals Chelating Agent available from Dow
12. Xiameter MEM-1872 Emulsion available from Dow (sufficiently low particle size to be clear in shampoo compositions at the levels used)
13. Octopirox available from Clariant
14. Zinc Pyrithione available from Lonza
15. Rheocare TTA available from BASF
16. Citric Acid USP Anhydrous Fine Granular available from Archer Daniels Midland Company

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A stable shampoo composition comprising:
a. a surfactant system comprising:
i. about 3% to about 35% of an anionic surfactant;
ii. about 5% to about 15% of an amphoteric surfactant;
b. about 0.01% to about 2% of a cationic polymer having a charge density of about 1.7 meq/g to about 2.1 meq/g; and
c. about 1% to about 1.5% inorganic salt, wherein the composition is substantially free of sulfated surfactants.

2. The composition of claim 1, wherein the shampoo composition comprises a %T of greater than about 80.

3. The composition of claim 1, wherein the shampoo composition lacks in situ coacervate, as determined by the Microscopy Method to Determine Lack of In Situ Coacervate.

4. The composition of claim 1, wherein the ratio of the anionic to the amphoteric surfactant is about 0.5:1 to about 1.5:1.

5. The composition of claim 1, comprising a pH of about 5 to 6.5.

6. The composition of claim 1, wherein the anionic surfactant is selected from sodium, ammonium or potassium salts of isethionates; sodium, ammonium or potassium salts of sulfonates; sodium, ammonium or potassium salts of ether sulfonates; sodium, ammonium or potassium salts of sulfosuccinates; sodium, ammonium or potassium salts of sulfoacetates; sodium, ammonium or potassium salts of glycinates; sodium, ammonium or potassium salts of sarcosinates; sodium, ammonium or potassium salts of glutamates; sodium, ammonium or potassium salts of alaninates; sodium, ammonium or potassium salts of carboxylates; sodium, ammonium or potassium salts of taurates; sodium, ammonium or potassium salts of phosphate esters; and combinations thereof.

7. The composition of claim 1 wherein the cationic polymer has a weight average molecular weight of about 300,000 g/mol to about 3,000,000 g/mol.

8. The composition of claim 7, wherein the cationic polymer is selected from cationic guars, cationic cellulose, cationic synthetic homopolymers, cationic synthetic copolymers, and combinations thereof.

9. The composition of claim 8, wherein the cationic polymer is selected from hyroxypropyltrimonium guar, Polyquaternium 10, and combinations thereof.

10. The composition of claim 1, wherein the inorganic salt is selected from sodium chloride, potassium chloride, sodium sulfate, ammonium chloride, sodium bromide, and combinations thereof.

11. The composition of claim 1, wherein the amphoteric surfactant is selected from betaines, sultaines, hydroxysultanes, amphohydroxypropyl sulfonates, alkyl amphoactates, alkyl amphodiacetates, and combination thereof.

12. The composition of claim 1, further comprising an antidandruff agent.

13. The composition of claim 12, wherein the antidandruff agent is selected from piroctone olamine, zinc pyrithione, and combinations thereof.

14. The composition of claim 1, having a viscosity about 3000 cP to about 20,000 cP.

15. The composition of claim 1, wherein the composition is composition is substantially free of silicones.

16. The composition of claim 1, wherein the composition consists of 9 or fewer ingredients.

17. The composition of claim 1, wherein the composition is substantially free of viscosity modifiers other than the inorganic salt.

18. A method for cleaning hair comprising:
a. providing the shampoo composition of claim 1;
b. dispensing the shampoo composition into a palm or a cleaning implement;
c. applying the shampoo composition onto wet hair and massaging the shampoo composition across the hair and scalp; wherein the shampoo composition is diluted forming a coacervate that is deposited onto the hair;
d. rinsing the shampoo composition from the hair.

19. A stable shampoo composition comprising:
a. a surfactant system comprising:
i. about 3% to about 35% of an anionic surfactant selected from isethionates, sarcosinates, and combinations thereof;
ii. about 5% to about 15% of an amphoteric surfactant selected from cocamidopropyl betaine, lauramidopropyl betaine, and combinations thereof;
wherein the ratio of the anionic to the amphoteric surfactant is about 0.5:1 to about 1.5:1;
b. about 0.01% to about 2% of a cationic polymer having a charge density of about

1. 7 meq/g to about 2.1 meq/g; wherein the cationic surfactant is selected from hyroxypropyltrimonium guar, Polyquaternium 10, and combinations thereof;
c. about 1% to about 1.5% sodium chloride;
wherein the composition is substantially free of sulfated surfactants;
wherein the shampoo composition lacks in situ coacervate, as determined by the Microscopy Method to Determine Lack of In Situ Coacervate.
